(19) United States
(12) Patent Application Publication
(10) Pub. No.: US 2018/0360321 A1
(43) Pub. Date: Dec. 20, 2018

(54) PHOTOACOUSTIC APPARATUS, CODING APPARATUS, AND INFORMATION PROCESSING APPARATUS

(71) Applicant: CANON KABUSHIKI KAISHA, Tokyo (JP)

(72) Inventor: Yukio Furukawa, Sagamihara-shi (JP)

(21) Appl. No.: 16/004,123

(22) Filed: Jun. 8, 2018

(30) Foreign Application Priority Data
Jun. 14, 2017 (JP) 2017-116919

(51) Int. Cl. A61B 5/00; A61B 6/00; A61B 8/08; A61B 8/00

(52) U.S. Cl. CPC A61B 5/0095; A61B 6/4417; A61B 8/4416; A61B 6/4411; A61B 8/5207

(57) ABSTRACT

A photoacoustic apparatus includes a light irradiation unit configured to irradiate a first irradiation region of a subject with first intensity modulated light corresponding to a first coding sequence and to irradiate a second irradiation region of the subject with second intensity modulated light corresponding to a second coding sequence, a reception unit configured to receive photoacoustic waves generated when the subject is irradiated with the first and the second intensity modulated light and output a first signal, and a processing unit configured to perform decoding processing on the first signal based on information on the first and the second coding sequences to acquire a first decoded signal corresponding to the first irradiation region or a second decoded signal corresponding to the second irradiation region.

### BACKGROUND

### Field of the Disclosure

The present disclosure relates to a photoacoustic apparatus using a photoacoustic effect.

### Description of the Related Art

In medical field, active studies have been made on optical imaging apparatuses for irradiating a subject with light and generating images based on subject internal information obtained through irradiation light. Photo Acoustic Tomography (PAT) is one of optical imaging techniques. In PAT, an optical imaging apparatus irradiates a subject with light generated by a light source, and detects a sound wave generated at a tissue that absorbed the energy of light propagated and diffused within the subject. This phenomenon of sound wave generation is referred to as a photoacoustic effect, and the sound wave generated is referred to as a photoacoustic wave. The sound wave is generally an ultrasonic wave.

“Dedicated 3D photoacoustic breast imaging”, Kruger R A, Kuzmiak C M, Lam R B, Reinecke D R, Del Rio S P, Steed D., Med Phys. 2013; 40:113301 (herein after, referred to as Non-patent Document 1) discusses a technique for irradiating a certain region of a subject with light and, after completing the reception of a photoacoustic wave generated due to light irradiation, irradiating another region of the subject with light, and starting receiving a photoacoustic wave that is subsequently generated. Further, the non-patent Document 1 discusses a technique for reconstructing image data based on photoacoustic waves generated within the subject through light irradiation.

The technique discussed in “Dedicated 3D photoacoustic breast imaging”, Kruger R A, Kuzmiak C M, Lam R B, Reinecke D R, Del Rio S P, Steed D., Med Phys. 2013; 40:113301 makes it possible to separate reception signals corresponding to respective irradiation regions. However, this method limits the number of times of light irradiation per unit time, resulting in a decrease in signal-to-noise (S/N) ratio of reception signals of photoacoustic waves acquired per unit time.

### SUMMARY

The present disclosure is directed to separating reception signals corresponding to respective irradiation regions while restraining the decrease in S/N ratio of reception signals of photoacoustic waves acquired per unit time in a photoacoustic apparatus using photoacoustic waves generated through a plurality of times of light irradiation.

According to an aspect of the present invention, a photoacoustic apparatus includes a light irradiation unit configured to irradiate a first irradiation region of a subject with first intensity modulated light corresponding to a first coding sequence, and to irradiate a second irradiation region of the subject with second intensity modulated light corresponding to a second coding sequence, a reception unit configured to receive photoacoustic waves generated when the subject is irradiated with the first intensity modulated light and the second intensity modulated light, and to output a first signal, and a processing unit configured to perform decoding processing on the first signal based on information on the first and the second coding sequences to acquire a first decoded signal corresponding to the first irradiation region or a second decoded signal corresponding to the second irradiation region.

Further features of the present invention will become apparent from the following description of exemplary embodiments with reference to the attached drawings.

### BRIEF DESCRIPTION OF THE DRAWINGS

FIGS. 1A and 1B are diagrams schematically illustrating light intensities corresponding to positive and negative coding elements and reception signals of photoacoustic waves.

FIGS. 2A, 2B, and 2C are diagrams schematically illustrating light intensities corresponding to coding sequences and reception signals of photoacoustic waves.

FIG. 3 is a block diagram illustrating a photoacoustic apparatus according to an exemplary embodiment.

FIG. 4 is a block diagram illustrating a computer and peripheral components according to an exemplary embodiment.

FIGS. 5A, 5B, and 5C are diagrams illustrating characteristics of a semiconductor laser.

FIGS. 6A, 6B, and 6C are diagrams illustrating reception signals corresponding to a positive coding element.

FIG. 7 is a sequence diagram illustrating a coding sequence according to a first exemplary embodiment.

FIG. 8 a diagram illustrating an arrangement of irradiation regions and a reception unit according to the first exemplary embodiment.

FIGS. 9A, 9B, 9C, and 9D are diagrams illustrating drive currents and reception signals according to the first exemplary embodiment.

FIG. 10 is a diagram illustrating a reception signal in consideration of noise according to the first exemplary embodiment.

FIGS. 11A, 11B, 11C, and 11D are diagrams illustrating other drive currents and reception signals according to the first exemplary embodiment.

FIG. 12 is a diagram illustrating another reception signal in consideration of noise according to the first exemplary embodiment.

FIGS. 13A, 13B, and 13C are diagrams illustrating decoded signals according to the first exemplary embodiment.

FIGS. 14A, 14B, and 14C are diagrams illustrating other decoded signals according to the first exemplary embodiment.

FIG. 15 is a diagram illustrating configurations of drive units according to the first exemplary embodiment.

FIG. 16 is a block diagram illustrating a photoacoustic apparatus according to a second exemplary embodiment.

FIG. 17 is a diagram illustrating an arrangement of irradiation regions and a reception unit according to the second exemplary embodiment.

FIG. 18 is a sequence diagram illustrating a coding sequence according to the second exemplary embodiment.

FIGS. 19A, 19B, 19C, and 19D are diagrams illustrating reception signals according to the second exemplary embodiment.

FIGS. 20A, 20B, 20C, and 20D are diagrams illustrating other reception signals according to the second exemplary embodiment.

FIGS. 21A, 21B, 21C, and 21D are diagrams illustrating still other reception signals according to the second exemplary embodiment.

FIGS. 22A, 22B, 22C, and 22D are diagrams illustrating yet still other reception signals according to the second exemplary embodiment.

FIGS. 23A, 23B, 23C, 23D, and 23E are diagrams illustrating decoded signals according to the second exemplary embodiment.

FIGS. 24A, 24B, 24C, 24D, and 24E are diagrams illustrating other decoded signals according to the second exemplary embodiment.

FIGS. 25A, 25B, 25C, 25D, and 25E are diagrams illustrating still other decoded signals according to the second exemplary embodiment.

### DESCRIPTION OF THE EMBODIMENTS

Exemplary embodiments of the present invention will be described below with reference to the accompanying drawings. However, apparatus configurations, image display formats, and sizes, materials, shapes, and relative arrangements of components described below are to be considered as illustrative, and the scope of the present invention is not to be limited to the following descriptions.

It is known that, when a material is irradiated with light, a sound wave (also referred to as a photoacoustic wave) is generated due to a photoacoustic effect. Generally, a larger absorption coefficient and more intense irradiation light generate a more intense photoacoustic wave. For example, when the skin surface is irradiated with light, a photoacoustic wave is generated not only from a blood vessel near the skin surface but also from a mole or hair on the skin surface. Typically, a photoacoustic wave having a higher sound pressure is generated from a mole or hair than from a blood vessel. Since light is attenuated with the propagation in a living body, a photoacoustic wave from a deep blood vessel is relatively weaker than a photoacoustic wave from a mole or hair. For these reasons, when imaging is performed, a reconstruction artifact of a photoacoustic wave generated from a mole or hair interferes with the image by a photoacoustic wave generated at a deep blood vessel, possibly making it difficult to recognize the image of the deep blood vessel. More specifically, it may be difficult to recognize the image of an optical absorber due to an artifact arising from light irradiation in a specific irradiation region. Therefore, in a case where photoacoustic waves are acquired from a plurality of division regions formed by dividing a irradiation region, if a certain division region includes a mole, hair, or other strong optical absorbers, which are not the observation target, reception signals of photoacoustic waves generated when the irradiation region is irradiated with light may not be used for image reconstruction.

When irradiating a plurality of irradiation regions with light, if the time period of light irradiation on a certain irradiation region overlaps with the time period of light irradiation on another irradiation region, it may be impossible to determine which irradiation region was irradiated with light to acquire a reception signal from generated photoacoustic waves. A certain technique is known to temporally separate reception signals, more specifically, to receive photoacoustic waves through light irradiation on a certain irradiation region and then receive photoacoustic waves through light irradiation on another irradiation region. However, in this technique in which a sufficient time period is allocated to receive photoacoustic waves generated through light irradiation on each irradiation region, the S/N ratio of reception signals of photoacoustic waves acquired per unit time will decrease.

The present inventor found out a technique for coding a reception signal by irradiating a certain irradiation region with intensity modulated light corresponding to a certain coding sequence and then irradiating another irradiation region with intensity modulated light corresponding to another coding sequence. If a reception signal of photoacoustic waves coded in this way is decoded by using information about the coding sequences used for coding, it becomes possible to acquire decoded signals respectively corresponding to a plurality of irradiation regions. Such coding and decoding processing makes it possible to separate reception signals corresponding to respective irradiation regions even when the irradiation periods of respective irradiation regions overlap with each other. Therefore, it is possible to improve the S/N ratio of reception signals of photoacoustic waves acquired per unit time.

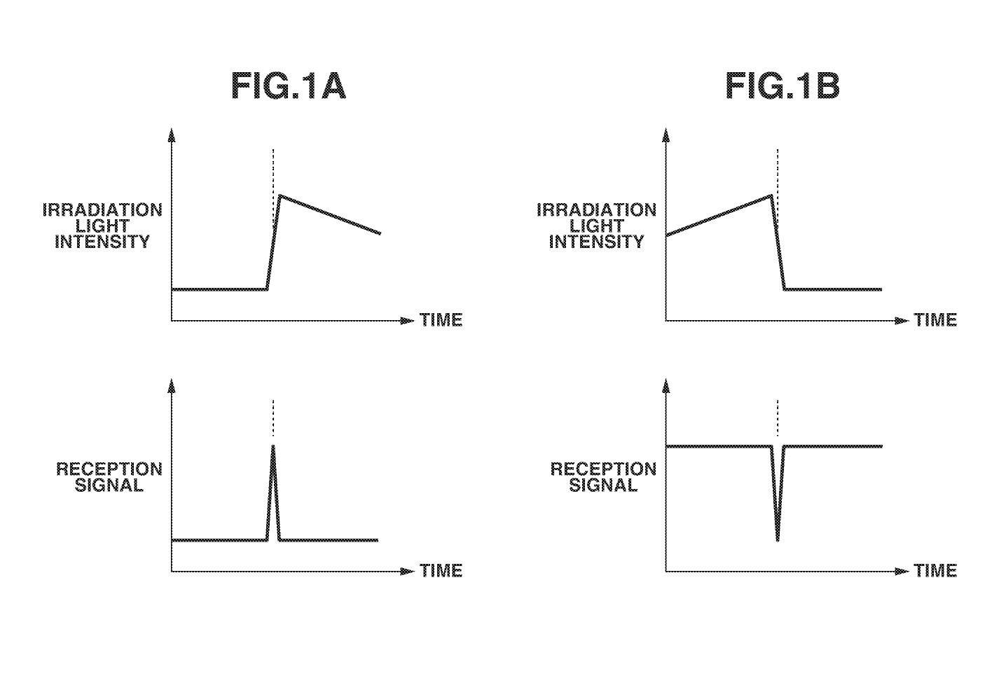

The following description is given of a coding method in a photoacoustic apparatus for processing reception signals of photoacoustic waves. The method performs coding processing based on coding sequences including the positive and negative coding elements through irradiation light control. FIGS. 1A and 1B are diagrams schematically illustrating intensities of irradiation light and temporal change of the levels of reception signals of photoacoustic waves generated due to the irradiation light. Typically, as illustrated in FIG. 1A, a positive temporal change in the irradiation light intensity enables acquiring a reception signal having a positive level. On the other hand, as illustrated in FIG. 1B, a negative temporal change in the irradiation light intensity enables acquiring a reception signal having a negative level. Further, there is a tendency that the levels of reception signals increase with increasing temporal change of the irradiation light intensity per unit time. Referring to FIGS. 1A and 1B, the time period of photoacoustic wave propagation from a sound source to a reception unit is ignored.

As illustrated in FIGS. 1A and 1B, the polarity of the level of a reception signal is controlled by controlling the polarity of the temporal change of the irradiation light intensity. More specifically, the polarity of coding elements configuring a coding sequence in coding processing is controlled by controlling the polarity of the temporal change of the irradiation light intensity. For example, when the coding element at the timing of light irradiation illustrated in FIG. 1A is defined as {1}, and the coding element at the timing of light irradiation illustrated in FIG. 1B is defined as {−1}, combining these light irradiations enables defining a coding sequence including the positive and negative coding elements. In the present specification, light for generating a photoacoustic wave corresponding to the positive coding element is referred to as “positive intensity modulated light”, and light for generating a photoacoustic wave corresponding to the negative coding element is referred to as “negative intensity modulated light”.

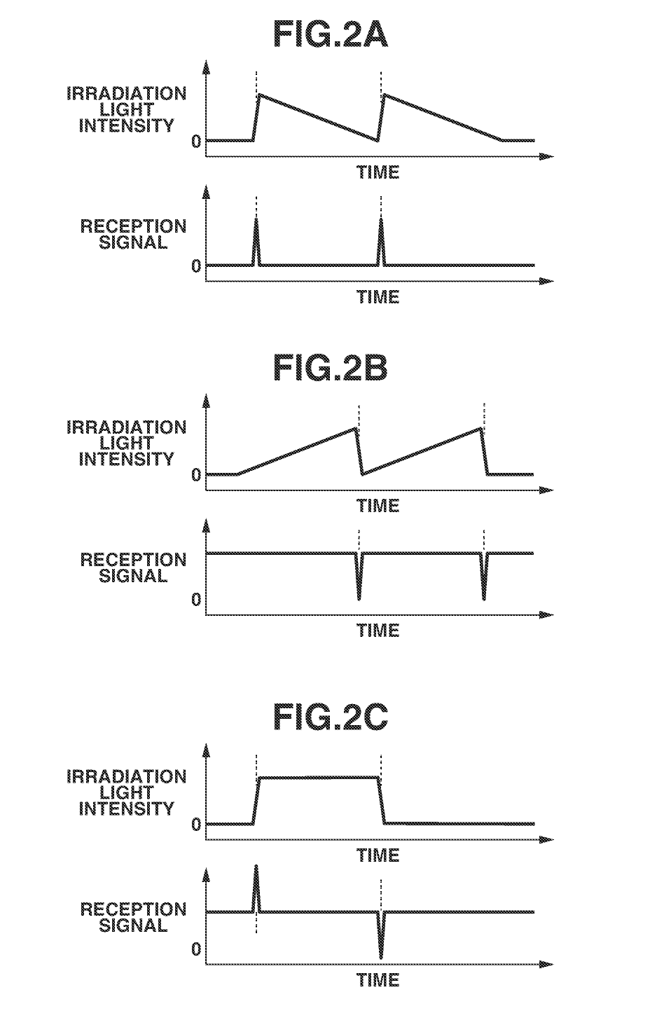

Hereinafter, examples of light irradiation sequences corresponding to several patterns of coding sequences are described with reference to FIGS. 2A, 2B, and 2C. Referring to FIGS. 2A, 2B, and 2C, a dotted line indicates the reference timing of each coding element.

FIG. 2A is a diagram schematically illustrating a temporal change of the irradiation light intensity corresponding to a coding sequence {1, 1} and the level of a reception signal of photoacoustic waves. The sequence of the irradiation light illustrated in FIG. 2A includes two successive waves of light (positive intensity modulated light) each of which steeply rises in a short time and then gently falls. The timing when the intensity steeply rises in a short time is adjusted to coincide with a reference timing corresponding to a positive coding element. For example, the timing of the center of the time period during which the intensity steeply rises in a short time can be matched with the reference timing. In this case, a large positive reception signal is acquired at the reference timing. This large positive reception signal serves as a signal corresponding to the positive coding element {1}.

FIG. 2B is a diagram schematically illustrating a temporal change of the intensity of irradiation light corresponding to a coding sequence {−1, −1} and the level of a reception signal of photoacoustic waves. The sequence of the irradiation light illustrated in FIG. 2B includes two successive waves of light (negative intensity modulated light) each of which gently rises with time and then steeply falls in a short time. The timing when the intensity steeply falls in a short time is adjusted to coincide with a reference timing corresponding to the negative coding element. More specifically, the timing of the center of the time period during which the intensity steeply falls in a short time is matched with the reference timing. In this case, a large negative reception signal is acquired at the reference timing. This large negative reception signal serves as a signal corresponding to the negative coding element {−1}.

FIG. 2C is a diagram schematically illustrating a temporal change of the intensity of irradiation light corresponding to a coding sequence {1, −1} and the level of a reception signal of photoacoustic waves. In the sequence of the irradiation light illustrated in FIG. 2C, a irradiation region is irradiated with the positive intensity modulated light illustrated in FIG. 2A and then the irradiation region is irradiated with the negative intensity modulated light illustrated in FIG. 2B. The irradiation timing is controlled so that the timing when the intensity of the positive intensity modulated light steeply rises coincides with the reference timing of the positive coding element {1}, and the timing when the intensity of the negative intensity modulated light steeply falls coincides with the reference timing of the negative coding element {−1}. In this case, a large positive reception signal and a large negative reception signal are acquired at respective reference timings.

The portion of the positive intensity modulated light which gently falls with time overlaps with the portion of the negative intensity modulated light which gently rises with time. As a result, the overlapped portion becomes a square waveform. When the positive and negative coding elements of a coding sequence adjoin each other, making the light intensity approximately constant between these reference timings prevents unnecessary photoacoustic waves from being generated in the time period. This enables accurately achieving coding processing through light irradiation. Although an example of a coding sequence {1, −1} has been described above with reference to FIG. 2C, the light intensity between reference timings may be similarly made approximately constant also in the case of a coding sequence {−1, 1}. Based on the reception bandwidth of a transducer for receiving photoacoustic waves, the temporal change of the light intensity between reference timings may be regarded as approximately constant as long as the temporal change falls within such a predetermined range that a photoacoustic wave having a frequency outside the reception bandwidth is generated. Referring to FIGS. 2A, 2B, and 2C, the time period of photoacoustic wave propagation from the sound source to the reception unit is ignored.

Performing light irradiation corresponding to a coding sequence including the positive and negative coding elements and then performing coding processing including the positive and negative coding elements in this way enable improving the decoding accuracy in decoding processing based on the coding sequence including the positive and negative coding elements. In particular, in a case of a semiconductor laser and a light emitting diode (LED) that output lower light intensity than a high power light source such as a solid-state laser, it is necessary to improve the S/N ratio of a reception signal by increasing the number of times of irradiation per unit time. In such a case, a decoded signal with a high S/N ratio can be accurately acquired by performing the following light irradiation and coding processing before completing reception of a previously generated photoacoustic wave based on a coding sequence including the positive and negative coding elements.

Sound waves generated due to the photoacoustic effect according to the present exemplary embodiment are typically ultrasonic waves including sound waves and acoustic waves. The present exemplary embodiment is applicable to a photoacoustic apparatus for acquiring image data based on photoacoustic waves generated due to the photoacoustic effect.

Conceptually, photoacoustic images acquired by the photoacoustic apparatus according to the present exemplary embodiment include all kinds of images resulting from photoacoustic waves generated through light irradiation. A photoacoustic image is image data representing a spatial distribution of at least one piece of subject information including the sound pressure of a photoacoustic wave (initial sound pressure), the optical absorption energy density, the optical absorption coefficient, and the density (oxygen saturation) of a material constituting a subject.

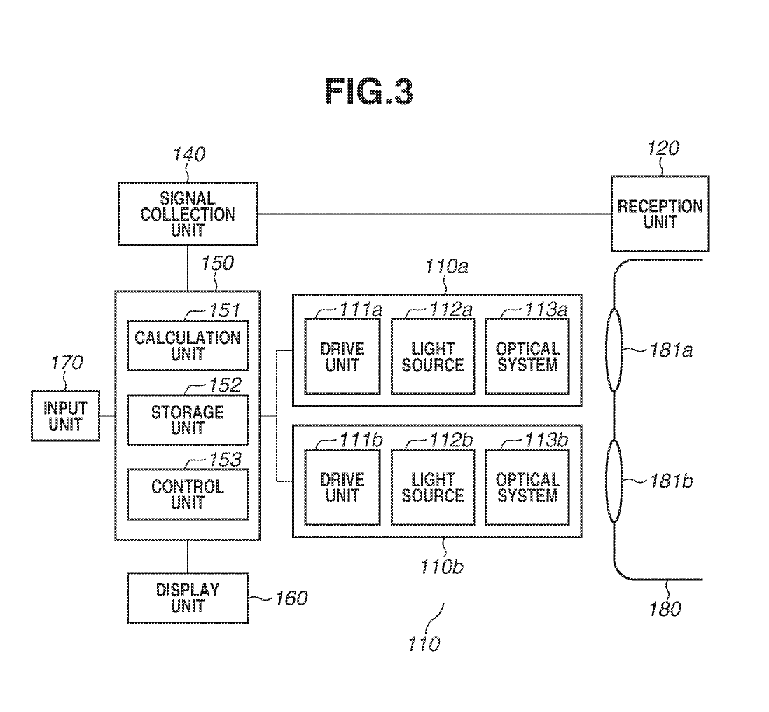

A configuration of the photoacoustic apparatus according to the present exemplary embodiment will be described below with reference to FIG. 3. FIG. 3 is a block diagram schematically illustrating the entire photoacoustic apparatus. The photoacoustic apparatus according to the present exemplary embodiment includes light irradiation unit 110 (a first light irradiation unit 110a and a second light irradiation unit 110b), a reception unit 120, a data acquisition unit 140, a computer 150, a display unit 160, and an input unit 170.

The first light irradiation unit 110a irradiates a first irradiation region 181a on a subject 180 with light, and a second light irradiation unit 110b irradiates a second irradiation region 181b on the subject 180 with light. As a result, sound waves are generated from the subject 180. A sound wave generated due to a photoacoustic effect resulting from light is also referred to as a photoacoustic wave. The reception unit 120 receives photoacoustic waves and outputs an electrical signal (photoacoustic signal) as an analog signal.

The data acquisition unit 140 converts the analog signal output from the reception unit 120 into a digital signal and outputs the digital signal to the computer 150. The computer 150 stores the digital signal output from the data acquisition unit 140 as signal data resulting from photoacoustic waves.

The computer 150 serving as a processing unit performs processing (described below) on the stored digital signal to generate image data representing a photoacoustic image. The computer 150 also performs image processing for display on the acquired image data and then outputs the image data to the display unit 160. The display unit 160 displays a photoacoustic image. A doctor or technician as a user can perform diagnosis by checking the photoacoustic image displayed on the display unit 160. The displayed image is stored in a memory in the computer 150 and a data management system connected with a modality via a network, based on a storage instruction from the user or the computer 150.

The computer 150 also performs drive control on components included in the photoacoustic apparatus. Further, the display unit 160 may display a graphical user interface (GUI) in addition to images generated by the computer 150. The input unit 170 is configured to allow a user to input information. By using the input unit 170, the user can start and end measurement, issue an instruction for storing a generated image, and perform other operations.

Each component of the photoacoustic apparatus according to the present exemplary embodiment will be described below in detail.

### <Light Irradiation Unit 110>

The light irradiation unit 110 includes the first light irradiation unit 110a for irradiating the first irradiation region 181a with light, and the second light irradiation unit 110b for irradiating the second irradiation region 181b with light.

The first light irradiation unit 110a includes a first light source 112a, and a first optical system 113a for guiding the light emitted from the first light source 112a to the first irradiation region 181a on the subject 180. The first light irradiation unit 110a includes a first drive unit 111a for controlling the driving of the first light source 112a.

The second light irradiation unit 110b includes a second light source 112b, and a second optical system 113b for guiding the light emitted from the second light source 112b to the second irradiation region 181b on the subject 180. The second light irradiation unit 110b includes a second drive unit 111b for controlling the driving of the second light source 112b.

Light generated by the first and the second light sources 112a and 112b may have a pulse width from 1 ns or more and 100 ns or less, and a wavelength of about 400 to 1600 nm. When imaging a blood vessel with a high resolution, light having a wavelength (400 nm or more and 700 nm or less) with a large absorption at a blood vessel may be used. When imaging a deep portion of a living body, light having a wavelength (700 nm or more and 1100 nm or less) with a small absorption typically at a background tissue (such as water and fat) of a living body may be used. A light source capable of emitting light having different wavelengths may be used.

The first and the second light sources 112a and 112b may be a laser or light emitting diode (LED), or may be a light source with a variable wavelength.

For example, as the first and the second light sources 112a and 112b, a semiconductor laser or LED capable of generating light following a sawtooth drive waveform (drive current) with a frequency of 1 MHz or higher is employable.

Lenses, mirrors, optical fibers, and other optical elements may be used for the first and the second optical systems 113a and 113b. When the subject 180 is a breast, a light emitting unit of each of the optical systems 113a and 113b may include a diffusion plate for diffusing light to irradiate the subject 180 with the increased beam diameter of pulsed light. On the other hand, in a photoacoustic microscope, the light emitting unit of each of the first and the second optical systems 113a and 113b may include lenses to irradiate the subject 180 with a focused beam to improve the resolution. The first and the second light irradiation units 110a and 110b may irradiate the subject 180 with light directly from the first and the second light sources 112a and 112b without having the optical systems 113a and 113b, respectively.

The first and the second drive units 111a and 111b each generate a drive current (current to be supplied to each the first and the second light sources 112a and 112b) for driving each of the first and the second light sources 112a and 112b. The first and the second drive units 111a and 111b may each use a power source capable of temporally changing the current to be supplied to each of the first and the second light sources 112a and 112b. The first and the second drive units 111a and 111b control the outputs of the first and the second light sources 112a and 112b, respectively, to generate light as illustrated in FIGS. 1A and 1B to implement coding processing. The first and the second drive units 111a and 111b may be controlled by a control unit 153 in a computer 150 (described below). The first and the second drive units 111a and 111b may each include a control unit for controlling the current value, and the control unit may control the supplied current. A relation between the drive current and the irradiation light intensity will be described below.

### <Reception Unit 120>

The reception unit 120 includes a transducer for receiving a sound wave and outputting an electrical signal, and a supporting member for supporting the transducer.

Constituent materials of the transducer include a piezoelectric ceramic material represented by titanic acid lead zirconate (PZT), and a macromolecule piezoelectricity film material represented by polyvinylidene fluoride (PVDF). Further, elements other than piezoelectric elements are also usable. For example, capacitive transducers (Capacitive Micro-machined Ultrasonic Transducers (CMUT) and transducers using a Fabry-Perot interferometer are usable. Any other types of transducers are also employable as long as the transducers are capable of receiving a sound wave and outputting an electrical signal. A signal acquired by a transducer is a time-resolved signal. More specifically, the amplitude of a signal acquired by a transducer represents a value based on the sound pressure (e.g., a value proportional to the sound pressure) received by the transducer at each time.

A photoacoustic wave includes frequency components of 100 kHz to 100 MHz. A transducer capable of detecting these frequencies is employable.

As a supporting member, a plurality of transducers may be arranged side by side in a plane or curved surface, which is referred to as a 1D array, 1.5D array, 1.75D array, or 2D array. When a plurality of transducers is arranged in a curved surface, this arrangement is also referred to as a three-dimensionally arranged transducer array (3D array).

The reception unit 120 may include an amplifier for amplifying a time series analog signal output from a transducer. Further, the reception unit 120 may also include an analog-to-digital (A/D) converter for converting a time series analog signal output from a transducer into a digital signal. In other words, the reception unit 120 may include the data acquisition unit 140 (described below).

To detect sound waves at various angles, ideally, transducers may be arranged so as to surround the entire circumference of the subject 180. However, if transducers cannot be arranged to surround the entire circumference of the subject 180 that has a large size, transducers may be arranged on a hemispherical supporting member to surround the entire circumference of the subject 180 as much as possible. It is only necessary to optimize the arrangement and the number of transducers, and the shape of the supporting member according to the subject 180. Any types of the reception unit 120 are applicable to the present exemplary embodiment.

The space between the reception unit 120 and the subject 180 may be filled with a medium that allows photoacoustic wave propagation. A material allowing sound wave propagation and acoustic characteristic matching at interfaces to the subject 180 and transducers is employable as this medium. For example, water and ultrasonic gel are employable as this medium.

In a case where the apparatus according to the present exemplary embodiment generates not only a photoacoustic image but also an ultrasonographic image through sound wave transmission and reception, a transducer may also function as a transmission unit for transmitting a sound wave. A transducer as a reception unit and a transducer as a transmission unit may be a single (common) transducer or different transducers.

The reception unit 120 may be a handheld type including a holding portion. Further, the reception unit 120 may be a mechanical scan type including a drive unit for mechanically moving a transducer 121.

### <Data Acquisition Unit 140>

The data acquisition unit 140 includes an amplifier for amplifying the electric signal (analog signal) output from the reception unit 120, and an A/D converter for converting the analog signal output from the amplifier into a digital signal. The data acquisition unit 140 may be constituted of a Field Programmable Gate Array (FPGA) chip. The digital signal output from the data acquisition unit 140 is stored in a storage unit 152 in the computer 150. The data acquisition unit 140 is also referred to as a Data Acquisition System (DAS). In the present disclosure, electric signals conceptually include analog and digital signals. The data acquisition unit 140 is connected with light detection sensors attached to light emission units of the light irradiation unit 110, and may start processing in synchronization with the light emission from the light irradiation unit 110. Alternatively, the data acquisition unit 140 may start the processing in synchronization with an instruction issued by using a freezing button as a trigger.

### <Computer 150>

The computer 150 serving as an information processing apparatus includes a calculation unit 151, a storage unit 152, and a control unit 153. The function of each component will be described below when processing flows are described below.

The calculation unit 151 having calculation functions includes a processor such as a central processing unit (CPU) and a graphics processing unit (GPU), and a calculation circuit such as a Field Programmable Gate Array (FPGA) chip. These units may include not only a single processor and a single calculation circuit but also a plurality of processors and a plurality of calculation circuits. The calculation unit 151 may receive various parameters such as a sound speed in the subject 180 and the sound velocity of a medium in which the acoustic wave propagates, sent from the input unit 170 and process the reception signals.

The storage unit 152 may include a non-transitory storage medium such as a read only memory (ROM), magnetic disk, and flash memory. The storage unit 152 may be a volatile medium such as a random access memory (RAM). The storage medium storing programs is a non-transitory storage medium. In addition, the storage unit 152 may include not only one storage medium but also a plurality of storage media.

The storage unit 152 can store image data representing photoacoustic images generated by the calculation unit 151, by using a method described below.

The control unit 153 includes a calculation element such as a CPU. The control unit 153 controls the operation of each component of the photoacoustic apparatus. The control unit 153 may control each component of the photoacoustic apparatus in response to instruction signals issued by various operations such as a measurement start operation from the input unit 170. The control unit 153 reads a program code stored in the storage unit 152 and controls the operation of each component of the photoacoustic apparatus.

The computer 150 may be a workstation designed for exclusive use. Components of the computer 150 may be configured as different hardware components. At least a part of components of the computer 150 may be configured as a single hardware component.

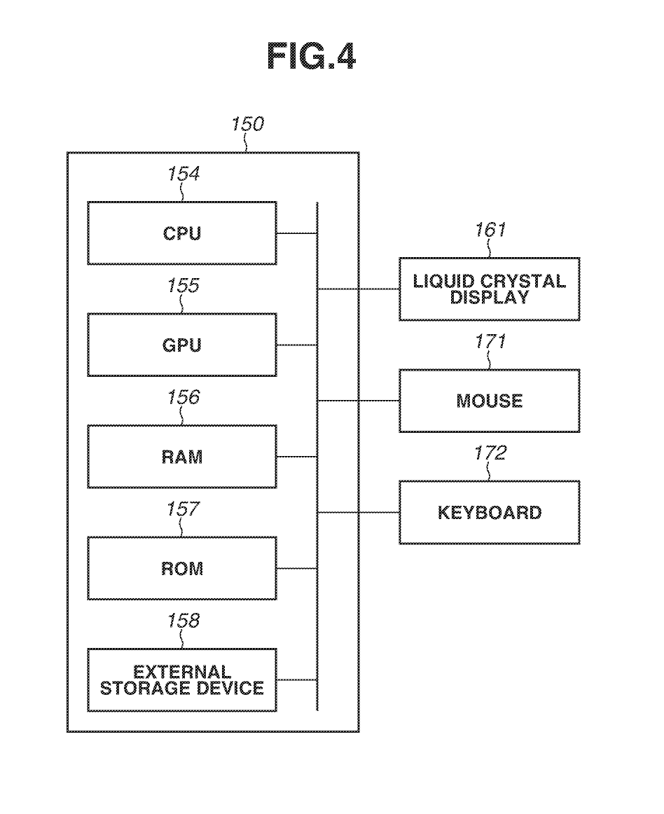

FIG. 4 illustrates a specific example of a configuration of the computer 150 according to the present exemplary embodiment. The computer 150 according to the present exemplary embodiment includes a CPU 154, a GPU 155, a RAM 156, a ROM 157, and an external storage device 158. Further, the computer 150 is connected with a liquid crystal display 161 as the display unit 160, and a mouse 171 and a keyboard 172 as the input unit 170.

The computer 150 and the reception unit 120 may be housed in a common housing. A computer housed in the housing may perform a part of signal processing, and a computer provided outside the housing may perform the remaining signal processing. In this case, the computers provided inside and outside the housing may be collectively referred to as the computer 150 according to the present exemplary embodiment. More specifically, hardware components configuring the computer 150 do not have to be stored in one housing.

### <Display Unit 160>

The display unit 160 is a display such as a liquid crystal display and an organic electro luminescence (EL). The display unit 160 is an apparatus for displaying images and numerical values at specific positions based on subject information acquired by the computer 150. The display unit 160 may display a GUI for operating an image and the apparatus. Before displaying the subject information, the display unit 160 or the computer 150 may perform image processing (luminance value adjustment) on the subject information.

### <Input Unit 170>

As the input unit 170, a user-operable operation console provided with a mouse and keyboard is employable. The display unit 160 may be provided with a touch panel and may be used as the input unit 170.

Components of the photoacoustic apparatus may be configured as different apparatuses or an integrated apparatus as one apparatus. In addition, at least a part of components of the photoacoustic apparatus may be integrated.

### <Subject 180>

The subject 180 does not constitute the photoacoustic apparatus. The subject 180 will be described below. The photoacoustic apparatus according to the present exemplary embodiment can be used for the purpose of diagnosis of malignant tumors and vascular diseases, and progress observation of chemical therapy for humans and animals. Therefore, the subject 180 is assumed to be a living body, more specifically, a diagnosis target portion such as the breast, each internal organ, vascular network, head, cervix, abdomen, and limbs including fingers and toes, of humans and animals. For example, if a human body is a measurement target, oxyhemoglobin or deoxyhemoglobin, a blood vessel containing a large amount of oxyhemoglobin or deoxyhemoglobin, and a new blood vessel formed near tumor may be used as a target optical absorber. Further, a plaque of a carotid wall may also be set as a target optical absorber. Pigments such as methylene blue (MB) and indocyanine green (ICG), golden particulates, a collection of these materials, and chemically modified materials introduced from outside may be used as an optical absorber. In addition, a puncture needle and an optical absorber applied to a puncture needle may be used as an observation target.

### <Reception Signal of Photoacoustic Wave Equivalent to Coding Element {1}>

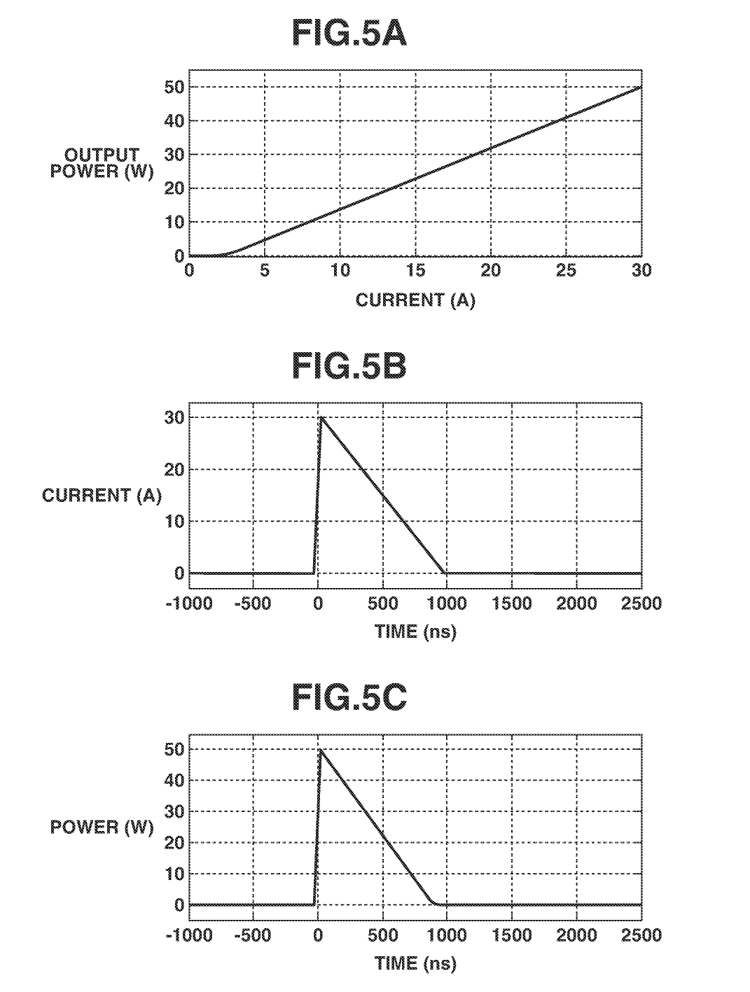

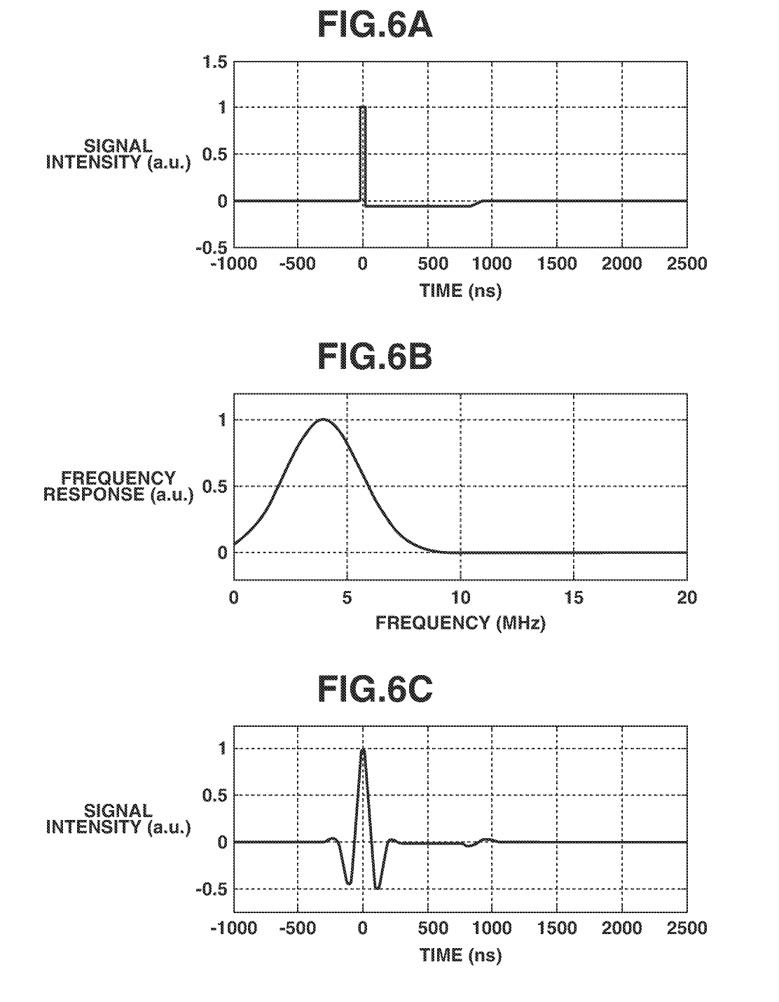

Now, irradiation light corresponding to each coding element and reception signals of photoacoustic waves when the photoacoustic apparatus according to the present exemplary embodiment is used, are considered. First, irradiation light corresponding to the coding element {1} and reception signals of photoacoustic waves will be described below with reference to FIGS. 5A to 5C or FIGS. 6A to 6C. The data illustrated in FIGS. 5A to 5C and the data illustrated in FIGS. 6A to 6C are data acquired through simulation.

FIG. 5A is a diagram illustrating the current-optical output characteristics of a semiconductor laser in a case where a wavelength of 808 nm is used as the first light source 112a or the second light source 112b. When the semiconductor laser has a threshold current of 1 A and a supplied current of 30 A, the optical output is 50 Watts. In a case of a semiconductor laser, the current-optical output characteristics typically provides an approximately linear relationship in a current region equal to or larger than a threshold value current. More specifically, in the case of a semiconductor laser, the temporal waveform of the supplied current provides a temporal waveform of the optical output (irradiation light intensity).

FIG. 5B illustrates a drive current (first drive current) for generating light corresponding to the positive coding element. The current value rises from 0 to 2 A in a time period of 50 ns and falls from 2 A to 0 A in a time period of 950 ns. More specifically, in the first drive current, the temporal change of the current at timings other than the timing corresponding to the positive coding element is smaller than the temporal change of the current at the timing corresponding to the positive coding element. As a result, in the positive intensity modulated light corresponding to the positive coding element, the temporal change of the light intensity at the reference timing corresponding to the positive coding element is larger than the temporal change of the light intensity at other timings.

FIG. 5C illustrates an optical output when a semiconductor laser is driven by the drive current illustrated in FIG. 5B. As described above, it is understood that the optical output is approximately linear with respect to the drive current.

FIG. 6A illustrates a reception signal when photoacoustic waves generated due to irradiating a point optical absorber with light are received by a transducer having an infinite reception bandwidth. This signal is equal to a result of time differentiation of the optical output curve illustrated in FIG. 5C. Thus, a large positive reception signal is acquired in accordance with the timing when the optical output steeply rises in a short time.

Actually, the transducer cannot have an infinite reception bandwidth, and therefore has certain frequency characteristics. FIG. 6B illustrates reception characteristics of a transducer having frequency characteristics including a center frequency of 4 MHz and a 6-dB bandwidth from 2 to 6 MHz. FIG. 6C illustrates a reception signal when photoacoustic waves generated due to the light irradiation illustrated in FIG. 5C are received by a transducer having reception characteristics illustrated in FIG. 6B. Thus, even in consideration of the reception characteristics of a transducer, a large positive reception signal is acquired in accordance with the timing when the optical output steeply rises in a short time. This large positive reception signal serves as the reception signal corresponding to the positive coding element (e.g., the coding element {1}).

In FIGS. 6A, 6B, and 6C, the time period of photoacoustic wave propagation from the sound source to the reception unit is ignored.

### <Photoacoustic Wave Equivalent to Coding Element {−1}>

Although detailed descriptions will be omitted, the reception signal of photoacoustic waves acquired when a semiconductor laser is driven by a drive current (second drive current), which is a drive current obtained by inverting the drive current, which is illustrated in FIG. 5B, around the time axis, becomes the reception signal obtained by inverting the reception signal illustrated in FIG. 6C around the time axis with the inverted signal level polarity. More specifically, in the second drive current, the temporal change of the current at timings other than the timing corresponding to the negative coding element is smaller than the temporal change of the current at the timing corresponding to the negative coding element. As a result, in the negative intensity modulated light corresponding to the negative coding element, the temporal change of the light intensity at the reference timing corresponding to the negative coding element is larger than the temporal change of the light intensity at other timings. The large negative reception signal acquired in this way serves as the reception signal corresponding to the negative coding element (e.g., the coding element {−1}).

### <Generation Flow of Photoacoustic Image>

A method for generating a photoacoustic image through coding and decoding processing (information processing method) by using the photoacoustic apparatus according to the present exemplary embodiment will be described below.

In step S1, the first light irradiation unit 110a irradiates the first irradiation region 181a of the subject 180 with first intensity modulated light coded with a first coding sequence. The second light irradiation unit 110b irradiates the second irradiation region 181b of the subject 180 with second intensity modulated light coded with a second coding sequence.

In step S2, a plurality of transducers included in the reception unit 120 receives photoacoustic waves generated due to the coded light and outputs first reception signals.

In step S3, the first light irradiation unit 110a irradiates the first irradiation region 181a of the subject 180 with third intensity modulated light coded with a third coding sequence. The second light irradiation unit 110b irradiates the second irradiation region 181b of the subject 180 with fourth intensity modulated light coded with a fourth coding sequence.

In step S4, the plurality of transducers included in the reception unit 120 receives photoacoustic waves generated due to the coded light and outputs second reception signals.

In step S5, the calculation unit 151 performs decoding processing on the first and the second reception signals output from the plurality of transducers to generate a decoded reception signal (decoded signal) for each transducer.

In step S6, the calculation unit 151 generates a photoacoustic image by using a plurality of decoded signals corresponding to the plurality of transducers.

As described above, the light irradiation unit 110 and the reception unit 120 configure a coding apparatus for generating coded signals. The coding apparatus performs light irradiation for coding, receives coded photoacoustic waves, and generates coded signals.

Specific coding and decoding methods will be described below in the present exemplary embodiment.

### <Reconstruction Method>

The calculation unit 151 is capable of performing back projection (simple back projection) of a plurality of decoded signals in the calculation space to generate image data. More specifically, the calculation unit 151 may convert decoded signals that are time signals into spatial distribution data. For example, the calculation unit 151 may perform delay and sum on a plurality of decoded signals to acquire linear image data in the depth direction (image data for one line). The calculation unit 151 may generate two- or three-dimensional image data by performing this processing on a plurality of lines. The calculation unit 151 may generate image data by performing envelope curve processing on the spatial distribution data acquired through delay and sum.

The Universal Back Projection (UBP) method is known as an image reconstruction technique for PAT. This method performs time differentiation on reception signals acquired by the reception unit 120 and performs back projection on polarity-inverted data to obtain a photoacoustic image. This method is applicable in a case where photoacoustic waves generated when impulsive pulsed light is radiated have a shape like the alphabetical character N called an N-shape.

On the other hand, it is conveniently understood that photoacoustic waves generated in the present exemplary embodiment are separated into a first half portion and a last half portion of the N-shape, and that the first half portion is a photoacoustic wave corresponding to the coding element {1}, and the last half portion is a photoacoustic wave corresponding to the coding element {−1}. Therefore, even if the UBP method is applied to a reception signal having undergone coding and decoding according to the present exemplary embodiment, a correct result cannot be acquired. Thus, according to the present exemplary embodiment, it is desirable that the calculation unit 151 performs delay and sum processing, without performing preprocessing in the UBP method, on the decoded reception signals and then performs back projection. According to the present specification, a reconstruction method for performing back projection, without performing preprocessing in the UBP method, on the decoded reception signals, is referred to as simple back projection. As a reconstruction algorithm for converting signal data into three-dimensional volume data, the back projection method in the time domain, the back projection method in the Fourier domain, the model base method (repetitive calculation method), and any other methods are applicable.

<Coding and Decoding Processing with Complementary Codes Applied>

Coding and decoding processing through light irradiation on two different regions, applying a complementary code pair, will be described below.

Assume that two coding sequences {aᵢ} and {cᵢ} with a code length of N (i=1 to N, N is the code length, and each coding element is 1 or −1). When the sum of the auto-correlation functions of the two coding sequences is 2N at the peak and 0 at all non-peak points, a pair of such coding sequences is referred to as a complementary code pair.

The auto-correlation function is represented by the following formula.

$${( a * a )}_{m} = {\sum }_{i = 1}^{N} {a}_{i + m} \cdot {a}_{i} \\ m = - N + 1 to N - 1 \qquad ( Formula 1 )$$

For example, a pair of {aᵢ}={1, 1} and {cᵢ}={1, −1} is a complementary code pair.

More specifically, (a*a)={1,2,1} and (c*c)={−1,2,−1} result in (a*a)+(c*c)={0,4,0}.

It is known that a complementary code pair exists when the code length is the n-th power of 2 or 5 times the n-th power of 2 (n is a natural number).

For a coding sequence pair, which is a complementary code pair, there exists another complementary code pair with which the sum of respective cross-correlation functions is 0. A relation between two complementary code pairs, where the sum of respective cross-correlation functions is 0, is conveniently referred to as a “complete orthogonal relation”.

A cross-correlation function is represented by the following formula.

$${( a * c )}_{m} = {\sum }_{i = 1}^{N} {a}_{i + m} \cdot {c}_{i} \\ m = - N + 1 to N - 1 \qquad ( Formula 2 )$$

For example, for a complementary code pair of a first coding sequence {aᵢ}={1,1} and a third coding sequence {cᵢ}={1,−1}, another complementary code pair of a second coding sequence {bᵢ}={1,−1} and a fourth coding sequence {dᵢ}={1,1} satisfies the “complete orthogonal relation”. More specifically, (a*b)={−1,0,1} and (c*d)={1,0,−1} satisfies (a*b)+(c*d)=0. In this case, (b*a)+(d*c)=0 is also satisfied.

For example, assume the following coding sequences with a code length of 8:

First coding sequence {aᵢ}={1,1,−1,1,−1,−1,−1,1}
Second coding sequence {bᵢ}={1,1,−1,1,1,1,1,−1}
Third coding sequence {cᵢ}={1,−1,−1,−1,−1,1,−1,−1}
Fourth coding sequence {dᵢ}={1,−1,−1,−1,1,−1,1,1}.
In this case, the following expressions result:
(a*a)={1,0,−3,0,−1,0,−1,8,−1,0,−1,0,−3,0,1}
(c*c)={−1,0,3,0,1,0,1,8,1,0,1,0,3,0,−1}
(a*a)+(c*c)={0,0,0,0,0,0,0,16,0,0,0,0,0,0,0}.
A pair of the first coding sequence {aᵢ} and the third coding sequence {cᵢ} is a complementary code pair. Likewise, the following expressions result:
(b*b)={−1,0,3,0,1,0,1,8,1,0,1,0,3,0,−1}
(d*d)={1,0,−3,0,−1,0,−1,8,−1,0,−1,0,−3,0,1}
(b*b)+(d*d)={0,0,0,0,0,0,0,16,0,0,0,0,0,0,0}.
A pair of the second coding sequence {bᵢ} and the fourth coding sequence {dᵢ} is also a complementary code pair. The following expressions also results:
(a*b)={−1,0,3,0,3,0,−1,0,−3,0,1,0,−3,0,1}
(c*d)={1,0,−3,0,−3,0,1,0,3,0,−1,0,3,0,−1}
(a*b)+(c*d)=0
(b*a)={1,0,−3,0,1,0,−3,0,−1,0,3,0,3,0,−1}
(d*c)={−1,0,3,0,−1,0,3,0,1,0,−3,0,−3,0,1}
(b*a)+(d*c)=0.
A pair of coding sequences {aᵢ} and {cᵢ} as a first complementary code pair, and a pair of coding sequences {bᵢ} and {dᵢ} as a second complementary code pair satisfy the “complete orthogonal relation”.

The use of coding sequence pairs satisfying the “complete orthogonal relation” in this way enables implementing the followings:
Assume a signal A coded with the first coding sequence {aᵢ} and a signal C coded with the third coding sequence {cᵢ}. The sum of a signal resulting from decoding the signal A with the first coding sequence {aᵢ} and a signal resulting from decoding the signal C with the third coding sequence {cᵢ} serves as a delta function.
The sum of a signal resulting from decoding the signal A with the second coding sequence {bᵢ} and a signal resulting from decoding the signal C with the fourth coding sequence {dᵢ} is 0.
Assume a signal B coded with the second coding sequence {bᵢ} and a signal D coded with the fourth coding sequence {dᵢ}. The sum of a signal resulting from decoding the signal B with the second coding sequence {bᵢ} and a signal resulting from decoding the signal D with the fourth coding sequence {dᵢ} serves as a delta function.
The sum of a signal resulting from decoding the signal B with the first coding sequence {aᵢ} and a signal resulting from decoding the signal D with the third coding sequence {cᵢ} is 0.

The above-described coding sequences are applied to a photoacoustic apparatus for performing light irradiation on two different regions. More specifically, the first irradiation region 181a is irradiated with light coded with the first coding sequence {aᵢ} and the third coding sequence {cᵢ}, and the second irradiation region 181b is irradiated with light coded with the second coding sequence {bᵢ} and the fourth coding sequence {dᵢ}.

In this case, even if light irradiation time periods overlap, decoding processing enables separately acquiring a signal resulting from the light emitted to the first irradiation region 181a and a signal resulting from the light emitted to the second irradiation region.

A case where light irradiation is performed on two different regions by using the photoacoustic apparatus illustrated in FIG. 3 will be described below. The first exemplary embodiment uses a semiconductor laser having a wavelength of 808 nm and a maximum optical output of 50 W for both the first light source 112a and the second light source 112b. The light emitted from the first light irradiation unit 110a irradiates the first irradiation region 181a on the subject 180, and the light emitted from the second light irradiation unit 110b irradiates the second irradiation region 181b on the subject 180.

The reception unit 120 includes a linear array composed of piezoelectric elements having the frequency characteristics including a center frequency of 4 MHz and a 6-dB bandwidth from 2 to 6 MHz. The gap between the reception unit 120 and the subject 180 is filled with ultrasonic gel for acoustic matching.

In the present exemplary embodiment, the complementary codes with a code length of 8 are used. More specifically the following complementary codes are used.

First coding sequence {aᵢ}={1,1,−1,1,−1,−1,−1,1}
Second coding sequence {bᵢ}={1,1,−1,1,1,1,1,−1}
Third coding sequence {cᵢ}={1,−1,−1,−1,−1,1,−1,−1}
Fourth coding sequence {dᵢ}={1,−1,−1,−1,1,−1,1,1}

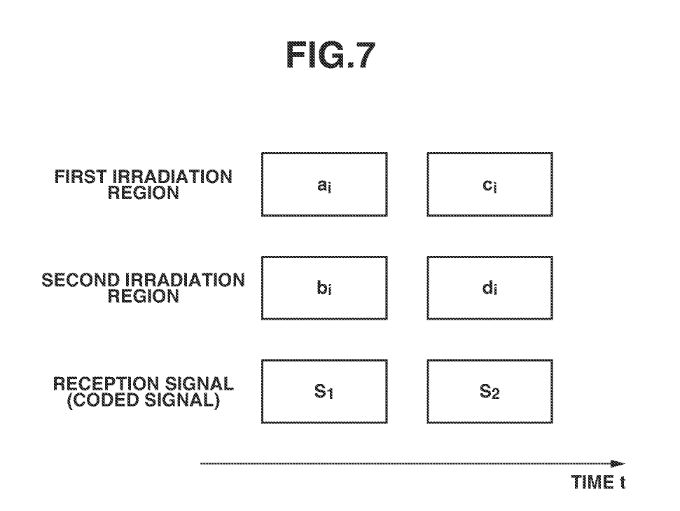

According to the present exemplary embodiment, in a sequence illustrated in FIG. 7, the computer 150 irradiates the first irradiation region 181a and the second irradiation region 181b with intensity modulated light, and acquires reception signals of generated photoacoustic waves, and then performs coding processing.

The first light irradiation unit 110a irradiates the first irradiation region 181a with intensity modulated light corresponding to the first coding sequence {aᵢ}, and the second light irradiation unit 110b irradiates the second irradiation region 181b with intensity modulated light corresponding to the second coding sequence {bᵢ}. The two intensity modulated light irradiations are performed at predetermined timings. The reception unit 120 receives photoacoustic waves generated due to the two intensity modulated light irradiations, and outputs a reception signal S₁.

Then, the first light irradiation unit 110a irradiates the first irradiation region 181a with intensity modulated light corresponding to the third coding sequence {cᵢ}, and the second light irradiation unit 110b irradiates the second irradiation region 181b with intensity modulated light corresponding to the fourth coding sequence {dᵢ}. The two intensity modulated light irradiations are performed at predetermined timings. Then, the reception unit 120 receives photoacoustic waves generated due to the intensity modulated light irradiation, and outputs a reception signal S₂.

Coding and decoding processing according to the present exemplary embodiment will be described in detail below.

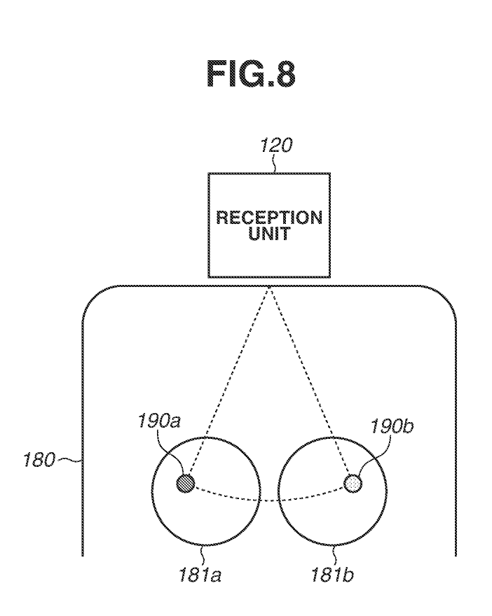

As illustrated in FIG. 8, a case is considered where the first irradiation region 181a is irradiated with intensity modulated light emitted from the first light irradiation unit 110a, and the second irradiation region 181b is irradiated with intensity modulated light emitted from the second light irradiation unit 110b. In addition, it is assumed that, in the subject 180, a first optical absorber 190a exists near the inner surface of the first irradiation region 181a, and a second optical absorber 190b having a smaller absorption coefficient than the first optical absorber 190a exists near the inner surface of the second irradiation region 181b. The ratio of the absorption coefficients of the first optical absorber 190a and the second optical absorber 190b with respect to light with a wavelength of 808 nm is assumed to be 1:0.5. Further, the distances from the reception unit 120 to respective optical absorbers are assumed to be equal.

The control unit 153 transmits information about the first coding sequence {aᵢ} to the first drive unit 111a, and transmits information about the second coding sequence {bᵢ} to the second drive unit 111b.

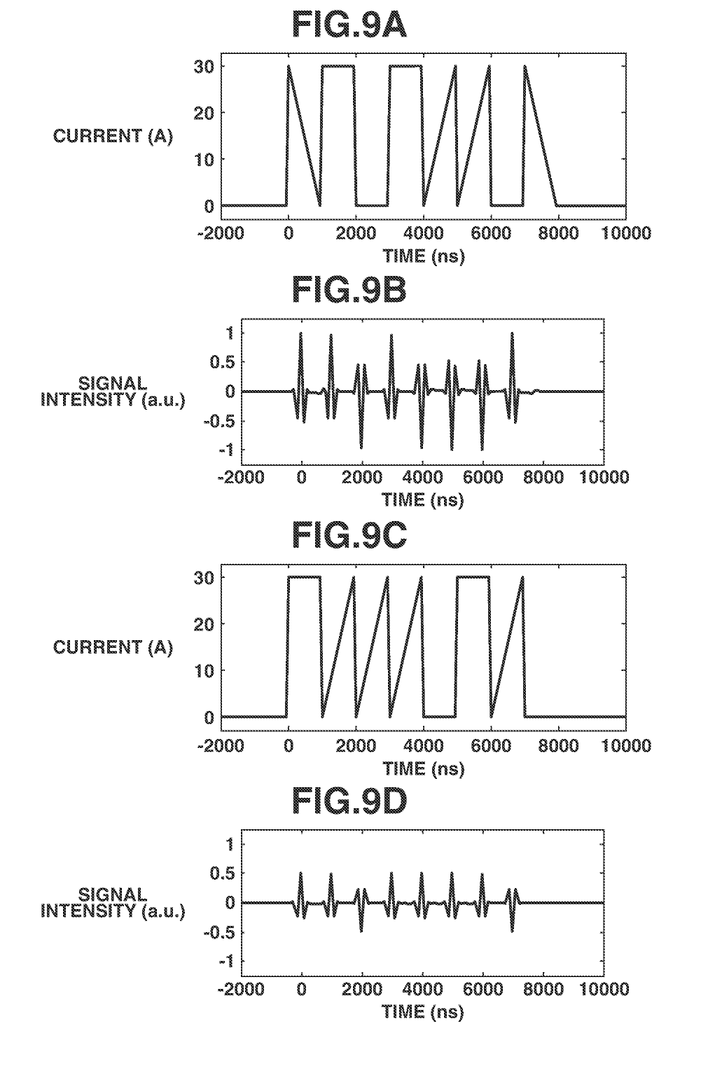

FIG. 9A illustrates a drive current generated by the first drive unit 111a based on the information about the first coding sequence {aᵢ}. The time interval between reference timings (equivalent to the cycle of coding element) is 1000 ns. The rising time and falling time of the first drive current corresponding to the positive coding element are 50 and 950 ns, respectively. The rising time and falling time of the second drive current corresponding to the negative coding element are 950 and 50 ns, respectively.

The first optical absorber 190a is irradiated with modulated light generated by the drive current illustrated in FIG. 9A, and the reception unit 120 receives the generated photoacoustic wave. A reception signal acquired in this way has a waveform as illustrated in FIG. 9B. Although the time actually shifts by the time period of photoacoustic wave propagation from the first optical absorber 190a to the reception unit 120, this time shift is ignored in FIG. 9B.

FIG. 9C illustrates a drive current generated by the second drive unit 111b based on the information about the second coding sequence {bᵢ}. Similar to FIG. 9A, the time interval between reference timings (equivalent to the cycle of coding element) is 1000 ns. The rising time and falling time of the first drive current corresponding to the positive coding element are 50 and 950 ns, respectively. The rising time and falling time of the second drive current corresponding to the negative coding element are 950 and 50 ns, respectively.

The second optical absorber 190b is irradiated with modulated light generated by the drive current illustrated in FIG. 9C, and the reception unit 120 receives the generated photoacoustic wave. A reception signal acquired in this way has a waveform as illustrated in FIG. 9D. Although the time actually shifts by the time period of photoacoustic wave propagation from the second optical absorber 190b to the reception unit 120, this time shift is ignored in FIG. 9D.

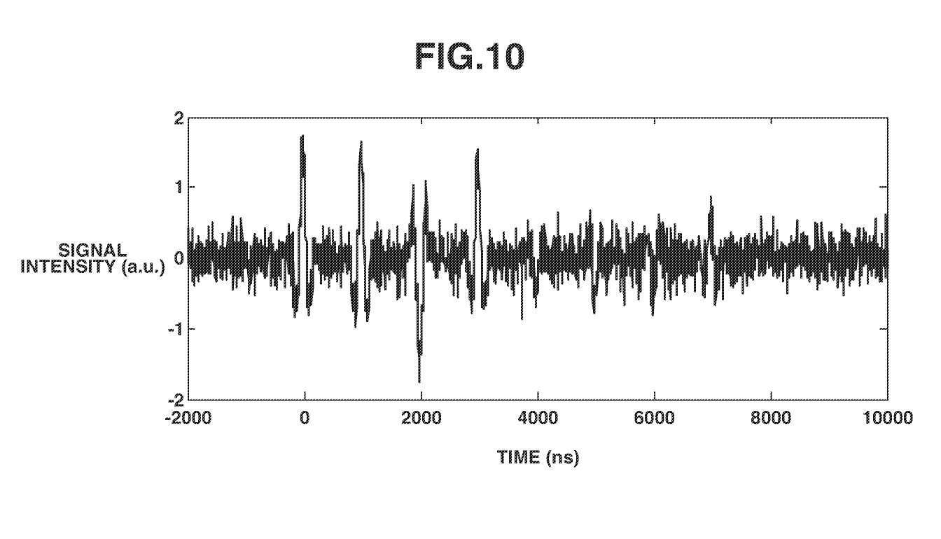

When the optical outputs of the first light source 112a and the second light source 112b are synchronized with each other (i.e., when these light sources emit light at approximately the same timing), the reception signal acquired when the reception unit 120 receives the generated photoacoustic waves is the sum of the signals illustrated in FIGS. 9B and 9D. An acquired reception signal S₁(t) is illustrated in FIG. 10. To describe the effect of noise suppression, noise with an average value of 0 and a standard deviation of 0.2 is added.

Subsequently, the control unit 153 transmits information about the third coding sequence {cᵢ} to the first drive unit 111a, and transmits information about the fourth coding sequence {dᵢ} to the second drive unit 111b.

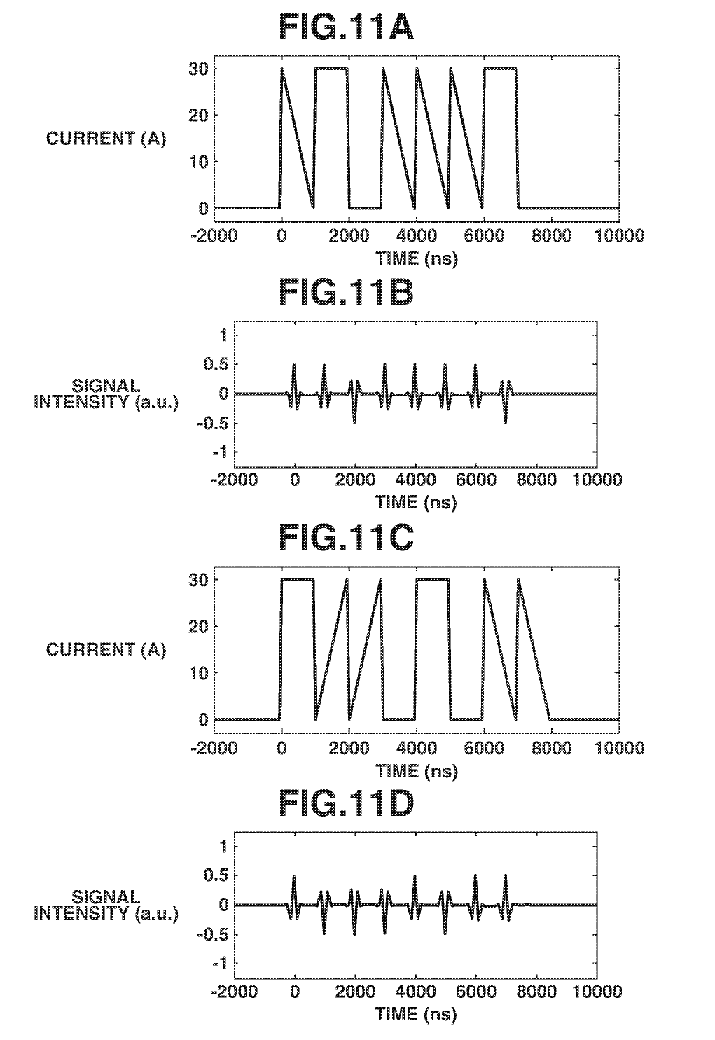

FIG. 11A illustrates a drive current generated by the first drive unit 111a based on the information about the third coding sequence {cᵢ}. The time interval between reference timings (equivalent to the cycle of coding element) is 1000 ns. The rising time and falling time of the first drive current corresponding to the positive coding element are 50 and 950 ns, respectively. The rising time and falling time of the second drive current corresponding to the negative coding element are 950 and 50 ns, respectively.

The first optical absorber 190a is irradiated with modulated light generated by the drive current illustrated in FIG. 11A, and the reception unit 120 receives the generated photoacoustic wave. A reception signal acquired in this way has a waveform as illustrated in FIG. 11B. Although the time actually shifts by the time period of photoacoustic wave propagation from the first optical absorber 190a to the reception unit 120, this time shift is ignored in FIG. 11B.

FIG. 11C illustrates a drive current generated by the second drive unit 111b based on the information about the fourth coding sequence {dᵢ}. Similar to FIG. 11A, the time interval between reference timings (equivalent to the cycle of coding element) is 1000 ns. The rising time and falling time of the first drive current corresponding to the positive coding element are 50 and 950 ns, respectively. The rising time and falling time of the second drive current corresponding to the negative coding element are 950 and 50 ns, respectively.

The second optical absorber 190b is irradiated with modulated light generated by the drive current illustrated in FIG. 11C, and the reception unit 120 receives the generated photoacoustic wave. A reception signal acquired in this way has a waveform as illustrated in FIG. 11D. Although the time actually shifts by the time period of photoacoustic wave propagation from the second optical absorber 190b to the reception unit 120, this time shift is ignored in FIG. 11D.

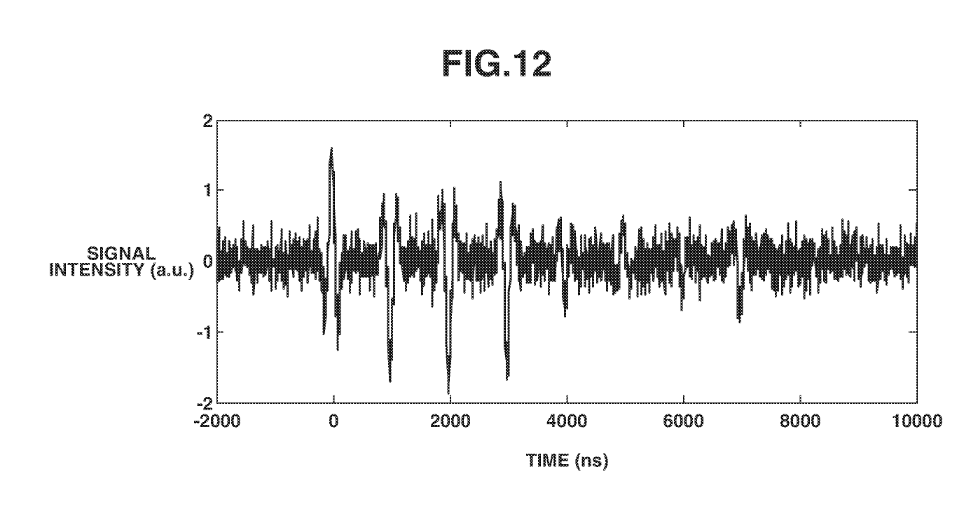

When the optical outputs of the first light source 112a and the second light source 112b are synchronized with each other (i.e., when these light sources emit light at approximately the same timing), the reception signal acquired when the reception unit 120 receives the generated photoacoustic waves is the sum of the signals illustrated in FIGS. 11B and 11D. An acquired reception signal S₂(t) is illustrated in FIG. 12. To describe the effect of noise suppression, noise with an average value of 0 and a standard deviation of 0.2 is added.

A method for decoding a coded reception signal performed by the calculation unit 151 in the computer 150 will be described below.

When Δt denotes the time interval between reference timings, the calculation unit 151 performs decoding processing on the reception signals S₁ and S₂ according to the Formula 3 to acquire a decoded signal DS₁(t) corresponding to the first irradiation region 181a. The calculation unit 151 also performs decoding processing on the reception signals S₁ and S₂ according to the Formula 4 to acquire a decoded signal DS₂(t) corresponding to the second irradiation region 181b.

$$\begin{matrix}{DS}_{1} ( t ) = {\sum }_{i = 1}^{N} {S}_{1} ( t + ( i - 1 ) Δ t ) \cdot {a}_{i} + {\sum }_{i = 1}^{N} {S}_{2} ( t + ( i - 1 ) Δ t ) \cdot {c}_{i} & ( Formula 3 ) \\ {DS}_{2} ( t ) = {\sum }_{i = 1}^{N} {S}_{1} ( t + ( i - 1 ) Δ t ) \cdot {b}_{i} + {\sum }_{i = 1}^{N} {S}_{2} ( t + ( i - 1 ) Δ t ) \cdot {d}_{i} & ( Formula 4 )\end{matrix}$$

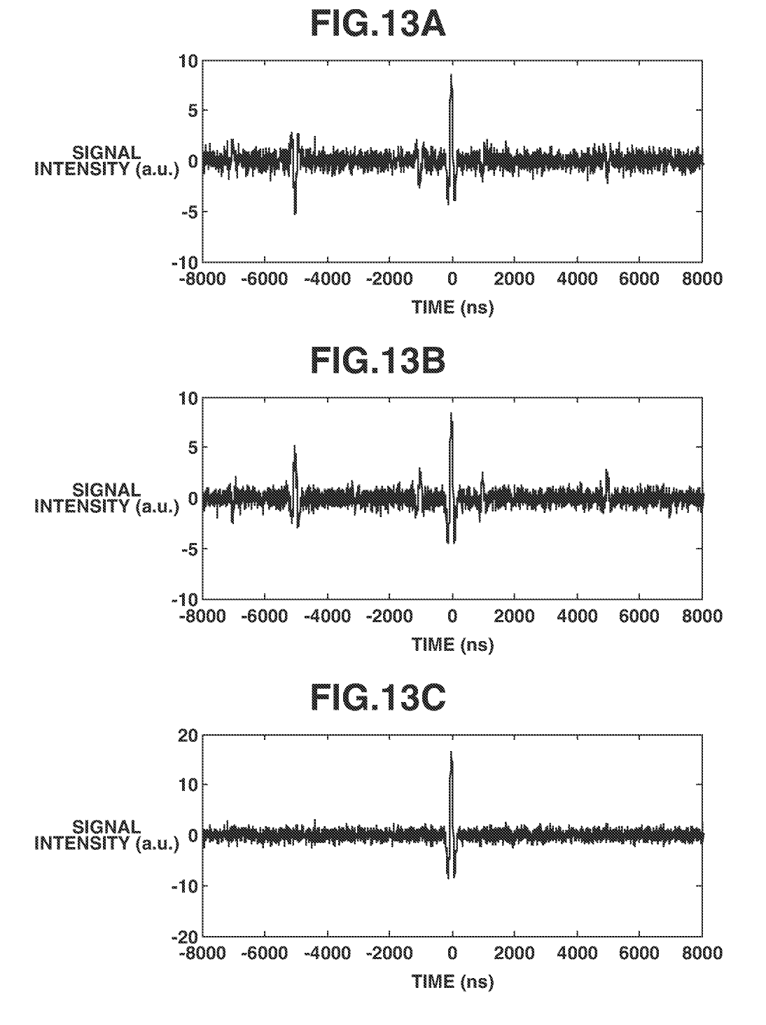

As a result of performing decoding processing of the first term of the right-hand side on the reception waveform illustrated in FIG. 10 (Formula 3), a decoded reception signal as illustrated in FIG. 13A can be acquired. As a result of performing decoding processing of the second term of the right-hand side of the Formula 3 on the reception waveforms illustrated in FIGS. 11A, 11B, 11C, and 11D, a decoded reception signal as illustrated in FIG. 13B can be acquired. Then, the sum of the signals illustrated in FIGS. 13A and 13B is the waveform (decoded signal DS₁) illustrated in FIG. 13C. More specifically, the peak value of the signal DS₁ decoded by the Formula 3 is increased 16 times the peak value of a reception signal acquired in one light irradiation, and side lobes are restrained to noise level or below.

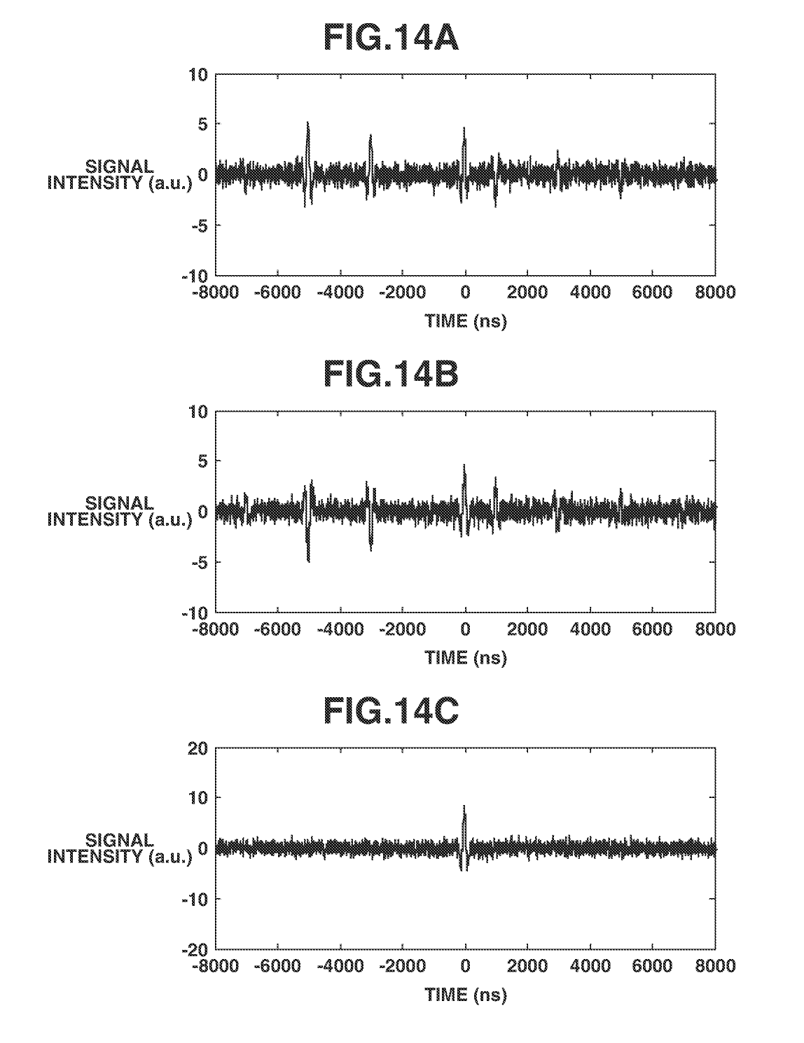

As a result of performing decoding processing of the first term of the right-hand side of the Formula 4 on the reception waveform illustrated in FIG. 10, a decoded reception signal as illustrated in FIG. 14A can be acquired. As a result of performing decoding processing of the second term of the right-hand side of the Formula 4 on the reception waveform illustrated in FIG. 12, a decoded reception signal as illustrated in FIG. 14B can be acquired. The sum of the signals illustrated in FIGS. 14A and 14B is the waveform (decoded signal DS₂) illustrated in FIG. 14C. More specifically, the peak value of the signal DS₂ decoded by the Formula 4 is increased 16 times the peak value of a reception signal acquired in one light irradiation, and side lobes are restrained to noise level or below.

When the signals illustrated in FIGS. 13C and 14C are compared, the ratio of the absorption coefficients of the absorbers is preserved. This means that information about the optical absorption of the subject 180 included in the reception signal is preserved even after coding and decoding processing is performed. Therefore, analyzing the decoded signals DS₁ and DS₂ enables obtaining an optical absorption coefficient distribution in the subject 180.

The noise level in the signal illustrated in FIG. 13C or 14C is restrained to a further extent than the noise level in the signal illustrated in FIG. 10 or 12. When a pair of coding sequences with a code length of 8 is used as in the present exemplary embodiment, the signal level increases 16 times and the noise level increases 4 times, and therefore the S/N ratio improves 4 times.

The calculation unit 151 generates a photoacoustic image by using decoded signals acquired in this way to enable obtaining a photoacoustic image with an improved S/N ratio. When the reception unit 120 includes a plurality of transducers, the reception unit 120 performs decoding processing on the reception signal output from each transducer to generate a decoded signal for each transducer. The calculation unit 151 can generate photoacoustic images based on the above-described reconstruction method by using the plurality of decoded signals corresponding to the plurality of transducers. According to the present exemplary embodiment, the computer 150 can generate photoacoustic images corresponding to the plurality of respective irradiation regions based on decoded signals corresponding to the plurality of respective irradiation regions. The computer 150 as a display control unit is able to display images for respective irradiation regions in a superimposed manner, in a parallelly arranged way, or in a switched way.

In this way, the present exemplary embodiment makes it possible to independently perform display control on images corresponding to the plurality of respective irradiation regions. Therefore, in an image corresponding to a certain irradiation region, even if there are many noise components resulting from the optical absorbers positioned on the surface of the subject 180, the image corresponding to a desired irradiation region with less noise components can be preferentially used for display.

Although, in the present exemplary embodiment, decoded signals corresponding to two respective irradiation regions are acquired, a decoded signal corresponding to at least one of the two irradiation regions only needs to be acquired. More specifically, according to the present exemplary embodiment, a decoded signal corresponding to at least one of a plurality of irradiation regions only needs to be acquired. Also in this case, the decoded signals corresponding to the desired irradiation regions can be acquired.

In the present exemplary embodiment, since, in the coding processing through light irradiation, light irradiation (negative intensity modulated light) corresponding to the negative coding element is performed, it is possible to accurately perform coding processing based on coding sequences including the negative coding element. Therefore, according to the present exemplary embodiment, the signal coded in this way can be accurately decoded through decoding (for example, decoding processing represented by the Formulas 3 and 4) based on coding sequences including the negative coding element. Thus, performing light irradiation corresponding to the negative coding element enables more accurately decoding the signal than in a case where light irradiation is not performed with a negative coding element of 0.

Further, in the present exemplary embodiment, by irradiating the subject 180 with the irradiation light from the first light source 112a and the irradiation light from the second light source 112b at almost the same timing to coincide the reception times with each other. As a result, the S/N ratio can be improved in a shorter time than in a case where photoacoustic waves resulting from the light of the two irradiation regions are received separately in time.

Although, in the present exemplary embodiment, the subject 180 is synchronously irradiated with light of the two irradiation regions, light irradiation at the same timing is not necessarily required. However, to shorten the measurement time, it is desirable to at least partially overlap the reception periods of photoacoustic waves resulting from the light of the two irradiation regions.

When photoacoustic waves resulting from the light of the two irradiation regions are received separately in time, a movement of the subject 180 during the reception time period causes a time shift in a signal. On the other hand, the method according to the present exemplary embodiment can reduce the time shift in a signal caused by a movement of the subject 180, by overlapping the time periods of light irradiations to the two irradiation regions.

An upper limit of the time interval between reference timings of coding elements according to the present exemplary embodiment will be described below.

The time required for one reception signal acquisition is equal to the time required for the photoacoustic wave generated at the furthest portion (viewed from the reception unit 120) in the observation region of the subject 180 to reach the reception unit 120. This required time is referred to as T_{tof}.

In the present exemplary embodiment, two coding sequences with a code length of 8 are used in one irradiation region. Therefore, in a decoded reception signal, the signal level increases 16 times and the noise level increases 4 times, and thus the S/N ratio improved 4 times.

To obtain the same improvement in the S/N ratio by using a generally known method for radiating impulsive pulsed light to generate photoacoustic waves, it is necessary to simply acquire a reception signal 16 times and then average the signals. If the time of light propagation in the subject 180 is ignored since the time is short, the time required to acquire a reception signal 16 times is 16T_{tof} when a common method is used. Since it is necessary to acquire reception signals for two irradiation regions, the required measurement time is 32T_{tof}.

The time required to acquire a reception signal corresponding to the first coding sequence {aᵢ} is the sum of the time required to radiate the light corresponding to the first coding sequence {aᵢ} and the time required for the photoacoustic wave generated due to the light corresponding to the last coding element to reach the reception unit 120. More specifically, the required time is 7Δt+T_{tof}. The time required to acquire each of the reception signals corresponding to the second to the fourth coding sequences {bᵢ}, {cᵢ}, and {dᵢ} is also equal. Therefore, when sequentially (serially) acquiring the reception signals corresponding to the first to the fourth coding sequences {aᵢ}, {bᵢ}, {cᵢ}, and {dᵢ}, the time required to acquire reception signals resulting from the light of the first irradiation region 181a and the light of the second irradiation region 181b is 28Δt+4T_{tof}.

In the present exemplary embodiment, the first and the second irradiation regions are simultaneously irradiated with light to simultaneously acquire respective reception signals. Thus, according to the present exemplary embodiment, the time required to acquire reception signals is 14Δt+2T_{tof}, which means that the time required to acquire reception signals is reduced in comparison with the method in which signals corresponding to respective irradiation regions are separated in time.

If the method according to the present exemplary embodiment provides a shorter time required to acquire reception signals than common methods, the method according to the present exemplary embodiment is more effective for improving the S/N ratio than common methods. This condition is 14Δt<30T_{tof}. If this condition is generalized by using a code length of N, the condition is represented by the following formula.

$$Δ t < \frac{2 N - 1}{N - 1} {T}_{tof} \qquad ( Formula 5 )$$

When N is large to a certain extent, it is desirable that Δt<2T_{tof}. More specifically, it is desirable that the time interval between reference timings is smaller than the twice the time required for the photoacoustic wave generated at the furthest portion (viewed from the reception unit 120) in the observation region of the subject 180 to reach the reception unit 120. For example, when the distance between the reception unit 120 and the furthest portion in the observation region of the subject 180 is 5 cm and the sound speed in the subject 180 is 1500 m/s, the time required for the photoacoustic wave generated at the furthest portion in the observation region of the subject 180 to reach the reception unit 180 is 33 μs. In this case, it is desirable that the time interval between reference timings is made shorter than 66 μs. In the case of a code length of 8, it is desirable that the time interval is made shorter than 71 μs based on the Formula 5. However, according to a target region specified via the input unit 170 by the user, the control unit 153 may change the time interval between reference timings to be shorter than the time required for the photoacoustic wave generated at the furthest portion to reach the reception unit 120. In addition, according to the sound speed in the subject 180 determined by a user instruction or calculation, the control unit 153 may change the time interval between reference timings to be shorter than the time required for the photoacoustic wave generated at the furthest portion to reach the reception unit 120.

### <Configuration of Drive Unit>

The drive current for generating positive intensity modulated light is referred to as a “first drive current”, and the drive current for generating negative intensity modulated light is referred to as a “second drive current”.

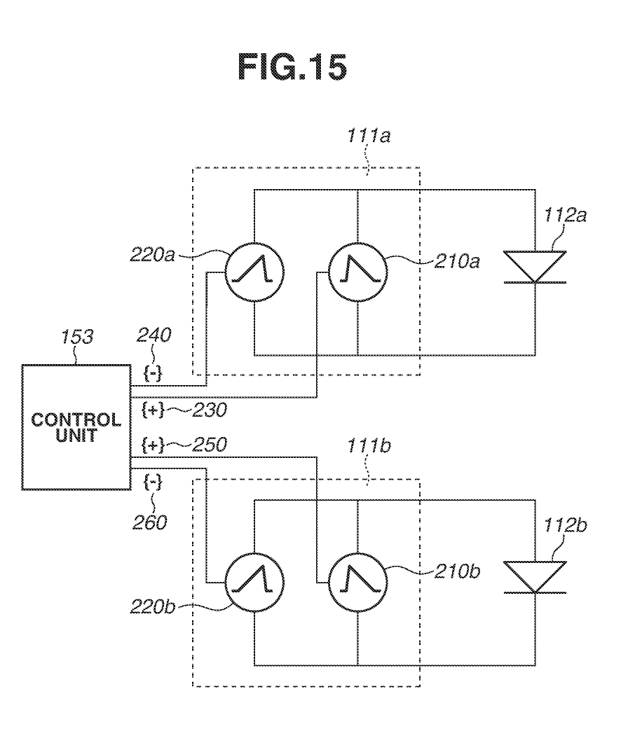

The first drive unit 111a or the second drive unit 111b may be configured of a power source capable of generating both the first and the second drive currents. Alternatively, the first drive unit 111a or the second drive unit 111b may include a first power source capable of generating the first drive current, and a second power source capable of generating the second drive current. An example in which the two drive currents are generated by different power sources will be described below with reference to FIG. 15.

The first drive unit 111a illustrated in FIG. 15 includes a first power source 210a capable of generating the first drive current, and a second power source 220a capable of generating the second drive current. The control unit 153 has a function of transmitting a first control signal 230 including 1 and 0 and a second control signal 240 including −1 and 0 to the first drive unit 111a.

For example, when performing light irradiation corresponding to the above-described first coding sequence {aᵢ}={1,1,−1,1,−1,−1,−1,1}, the control unit 153 separates a control signal into a first control signal {1,1,0,1,0,0,0,1} and a second control signal {0,0,−1,0,−1,−1,−1,0}, and transmits each signal to the first drive unit 111a. More specifically, the control unit 153 transmits the first control signal 230 to the first power source 210a and transmits the second control signal 240 to the second power source 220a.

The first power source 210a generates the first drive current in accordance with the timing of the coding element {1} of the first control signal, and zeros the current at the timing of the coding element {0} of the first control signal, or generates a current with which the photoacoustic wave generation is restrained. The second power source 220a generates the second drive current in accordance with the timing of the coding element {−1} of the second control signal, and zeros the current at the timing of the coding element {0} of the second control signal, or generates a current with which the photoacoustic wave generation is restrained. As a result, the first light source 112a is supplied with a current similar to the drive current (FIG. 9A) corresponding to the first coding sequence {aᵢ}.

The second drive unit 111b illustrated in FIG. 15 includes a third power source 210b capable of generating the first drive current and a fourth power source 220b capable of generating the second drive current. The control unit 153 has a function of transmitting a third control signal 250 including 1 and 0 and a fourth control signal 260 including −1 and 0 to the second drive unit 111b.

For example, when performing light irradiation corresponding to the above-described second coding sequence {bᵢ}={1,1,−1,1,1,1,1,−1}, the control unit 153 separates a control signal into a third control signal {1,1,0,1,1,1,1,0} and a fourth control signal {0,0,−1,0,0,0,0,−1}, and transmits each signal to the second drive unit 111b. More specifically, the control unit 153 transmits the third control signal 250 to the third power source 210b and transmits the fourth control signal 260 to the fourth power source 220b.

The third power source 210b generates the first drive current in accordance with the timing of the coding element {1} of the third control signal, and zeros the current at the timing of the coding element {0} of the third control signal, or generates a current with which the photoacoustic wave generation is restrained. The fourth power source 220 generates the second drive current in accordance with the timing of the coding element {−1} of the fourth control signal, and zeros the current at the timing of the coding element {0} of the fourth control signal, or generates a current with which the photoacoustic wave generation is restrained. As a result, the second light source 112b is supplied with a current similar to the drive current (FIG. 9C) corresponding to the third coding sequence {bᵢ}.

An apparatus including different power sources for respective drive currents can simplify the design of the first drive unit 111a or the second drive unit 111b to a further extent than in a case where different drive currents are generated by one power source. When using different power sources for respective drive currents, the apparatus provides high response when switching between different drive currents at a high speed. As a result, the subject 180 can be irradiated with light of different coding elements to overlap in time. This makes it possible to improve the light irradiation efficiency and acquiring decoded signals with a high S/N ratio in a short time.

Although, in the present exemplary embodiment, the maximum intensity of the peak optical output is equal for the first light source 112a and the second light source 112b, the setting is not limited thereto. According to the present exemplary embodiment, the levels of the coding elements {1} and {−1} in the first light source 112a need to be close to a certain extent, and the levels of the coding elements {1} and {−1} in the second light source 112b also need to be close to a certain extent. This means that the levels are close to such an extent that variations can be ignored through averaging. However, the level of the coding element {1} in the first light source 112a and the level of the coding element {1} in the second light source 112b do not need to be equal. For example, individual differences between respective light sources may differentiate the optical outputs at each timing even with the same supplied current. In this case, the supplied currents may be changed for respective light sources to equalize the maximum intensities of the optical outputs. Even when the maximum intensities of the peak optical outputs is different, the maximum intensities can be corrected by standardizing decoded reception signals with the maximum peak intensities of respective optical outputs. Alternatively, decoding processing may be performed after standardizing reception signals with the maximum intensities of respective optical outputs.

According to the present exemplary embodiment, the code length and the time interval between reference timings are not limited thereto, and suitable ones may be used so as to improve the S/N ratio according to the depth of the observation region in the subject 180 and the performance of a light source drive unit.

Although, in the present exemplary embodiment, two irradiation regions horizontally arranged are used, the arrangement of a plurality of irradiation regions may be in any form as long as mutually different regions are irradiated with light. For example, a plurality of irradiation regions may be formed in concentric ring shapes with different radii.

<Coding and Decoding Processing with Orthogonal Codes Applied>

Although two complementary code pairs satisfying the “complete orthogonal relation” exist, three or more complementary code pairs mutually satisfying the “complete orthogonal relation” do not exist. Therefore, the method according to the first exemplary embodiment is not applicable to three or more irradiation regions.

A second exemplary embodiment will be described below centering on coding and decoding processing when the above-described method is applied to three or more irradiation regions.

Four different coding sequences {aᵢ^{k}} (k=1 to 4, i=1 to 4) with a code length of 4, which are orthogonal to each other, are considered.

First coding sequence {a₁¹}={1,−1,−1,1}
Second coding sequence {aᵢ²}={1,−1,1,−1}
Third coding sequence {aᵢ³}={1,1, −1,−1}
Fourth coding sequence {aᵢ⁴}={1,1,1,1}
Further, four mutually different permutations {gₘ} (m=1 to 4, each element is 1 to 4) indicating the order of sequentially arranging these four coding sequences without duplication, are considered. First permutation {gᵢ}={{aᵢ¹},{aᵢ²}, {aᵢ³}, {aᵢ⁴}}
Second permutation {g₂}={{aᵢ²}, {aᵢ¹},{aᵢ⁴}, {aᵢ³}}
Third permutation {g₃}={{aᵢ³}, {aᵢ⁴}, {aᵢ¹},{aᵢ²}}
Fourth permutation {g₄}={{aᵢ⁴}, {aᵢ³}, {aᵢ²}, {aᵢ¹}}
The sum total of cross-correlation functions when the four coding sequences are arranged according to permutations gₚ and g_{q}, is considered. The sum total is represented by the following formula:

$${\sum }_{j = 1}^{4} ( {a}^{{g}_{p} ( j )} * {a}^{{g}_{q} ( j )} ) \qquad ( Formula 6 )$$

(p and q are integers from 1 to 4.)

When p=q, the Formula 6 represents the sum total of auto-correlation functions, i.e., 16 at the peak, and 0 at all non-peak points. For example, assume the following case:

(a¹*a¹)={1,−2,−1,4,−1,−2,1}
(a²*a²)={−1,2,−3,4,−3,2,−1}
(a³*a³)={−1,−2,1,4,1,−2,−1}
(a⁴*a⁴)={1,2,3,4,3,2,1}
In this case, the following formula is given at an arbitrary point p:

$${\sum }_{j = 1}^{4} ( {a}^{{g}_{p} ( j )} * {a}^{{g}_{p} ( j )} ) = ( {a}^{1} * {a}^{1} ) + ( {a}^{2} * {a}^{2} ) + ( {a}^{3} * {a}^{3} ) + ( {a}^{4} * {a}^{4} ) = \{ 0 , 0 , 0 , 1 , 6 , 0 , 0 , 0 \} \qquad ( Formula 7 )$$

When p≠q, the Formula 6 represents the sum total of cross-correlation functions, i.e., 0 at all points in all combinations where p≠q. For example, assume the following case where p=1 and q=2:

(a¹*a²)={−1,2,−1,0,1,−2,1}
(a²*a¹)={1,−2,1,0,−1,2,−1}
(a³*a⁴)={1,2,1,0,−1,−2,−1}
(a⁴*a³)={−1,−2,−1,0,1,2,1}
In this case, the following formula is given.

$${\sum }_{j = 1}^{4} ( {a}^{{g}_{1} ( j )} * {a}^{{g}_{2} ( j )} ) = ( {a}^{1} * {a}^{2} ) + ( {a}^{2} * {a}^{1} ) + ( {a}^{3} * {a}^{4} ) + ( {a}^{4} * {a}^{3} ) = \{ 0 , 0 , 0 , 0 , 0 , 0 , 0 \} \qquad ( Formula 8 )$$

Using coding sequence pairs having the above-described characteristics enables implementing the followings:
When coded signals are sequentially acquired according to four coding sequences in the order determined by a permutation gₚ, and the acquired signals are sequentially decoded according to four coding sequences in the order determined by the same permutation gₚ, the sum total of the decoded signals becomes a delta function.
When coded signals are sequentially acquired according to four coding sequences in the order determined by a permutation gₚ, and the acquired signals are sequentially decoded according to four coding sequences in the order determined by a permutation g_{q} different from the permutation gₚ, the sum total of the decoded signals becomes 0.

A case where the above-described coding sequences and a permutation indicating their order are applied to a photoacoustic apparatus for irradiating a plurality of mutually different irradiation regions with light, will be considered here. In this case, even if light irradiation time periods overlap, it is possible to separately acquire a signal resulting from the light of a certain irradiation region and a signal resulting from the light of another irradiation region.

A suitable relation between the number of irradiation regions, code length, and the number of coding sequences, which are orthogonal to each other, will be considered here. When the number of irradiation regions is 2 to 4, it is desirable that the code length is set to 4 and the number of codes which are orthogonal to each other is set to 4. When the number of irradiation regions is 5 to 8, it is desirable that the code length is set to 8 and the number of codes which are orthogonal to each other is set to 8. It is desirable that the number of coding sequences which are orthogonal to each other is set to the power of 2 equal to or larger than the number of irradiation regions.

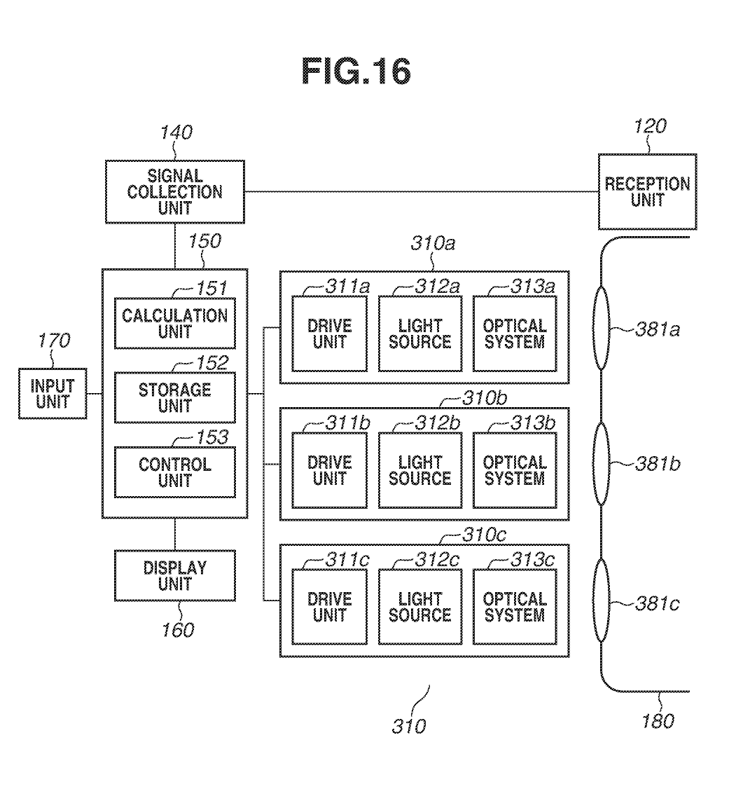

The present exemplary embodiment will be described below centering on a case where three different irradiation regions are irradiated with light by using a photoacoustic apparatus illustrated in FIG. 16. FIG. 16 is a block diagram schematically illustrating the entire photoacoustic apparatus. Members identical to those of the photoacoustic apparatus illustrated in FIG. 3 are assigned the same reference numerals, and redundant descriptions thereof will be omitted. The photoacoustic apparatus according to the present exemplary embodiment includes light irradiation units 310 (a first light irradiation unit 310a, a second light irradiation unit 310b, and a third light irradiation unit 310c), a reception unit 120, a data acquisition unit 140, a computer 150, a display unit 160, and an input unit 170.

The light irradiation units 310 include the first light irradiation unit 310a for irradiating a first irradiation region 381a with light, a second light irradiation unit 310b for irradiating a second irradiation region 381b with light, and a third light irradiation unit 310c for irradiating a third irradiation region 381c with light.

The first light irradiation unit 310a includes a first light source 312a, and a first optical system 313a for guiding the light emitted from the first light source 312a to the first irradiation region 381a on the subject 180. The first light irradiation unit 310a includes a first drive unit 311a for controlling the drive of the first light source 312a.

The second light irradiation unit 310b includes a second light source 312b, and a second optical system 313b for guiding the light emitted from the second light source 312b to the second irradiation region 381b on the subject 180. The second light irradiation unit 310b includes a second drive unit 311b for controlling the drive of the second light source 312b.

The third light irradiation unit 310c includes a third light source 312c, and a third optical system 313c for guiding the light emitted from the third light source 312c to the third irradiation region 381c on the subject 180. The third light irradiation unit 310c includes a third drive unit 311c for controlling the drive of the third light source 312c.

In the present exemplary embodiment, a semiconductor laser having a wavelength of 808 nm and a maximum optical output of 50 W as the first light source 312a, the second light source 312b, and the third light source 312c, is used.

A linear array composed of piezoelectric elements having the frequency characteristics including a center frequency of 4 MHz and a 6-dB bandwidth from 2 to 6 MHz is used as the reception unit 120 a. The gap between the reception unit 120 and the subject 180 is filled with ultrasonic gel for acoustic matching.

In the present exemplary embodiment, the following four coding sequences with a code length of 4, which are orthogonal to each other, are used:

First coding sequence {aᵢ¹}={1,−1,−1,1}
Second coding sequence {aᵢ²}={1,−1,1,−1}
Third coding sequence {aᵢ³}={1,1, −1,−1}
Fourth coding sequence {aᵢ⁴}={1,1,1,1}
Permutations assigned to the first, the second, and the third irradiation regions are as follows:
First permutation {gᵢ}={{aᵢ¹},{aᵢ²}, {aᵢ³}, {aᵢ⁴}}
Second permutation {g₂}={{aᵢ²}, {aᵢ¹},{aᵢ⁴}, {aᵢ³}}
Third permutation {g₃}={{aᵢ³}, {aᵢ⁴}, {aᵢ¹},{aᵢ²}}.

More specifically, the four coding sequences are assigned, in the order determined by the first permutation, to the light to be radiated to the first irradiation region 381a. The four coding sequences are assigned, in the order determined by the second permutation, to the light to be radiated to the second irradiation region 381b. The four coding sequences are assigned, in the order determined by the third permutation, to the light to be radiated to the third irradiation region 381c.

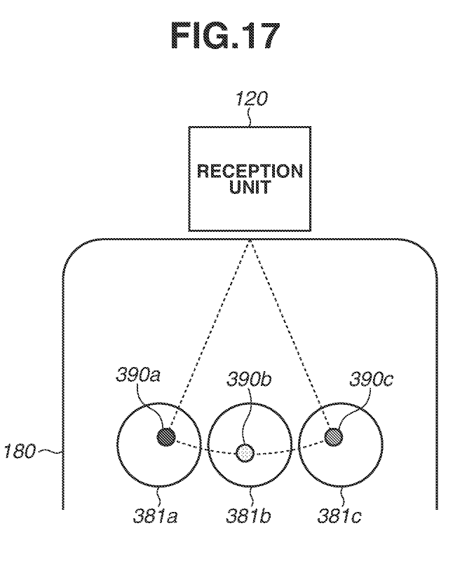

The flow of coding and decoding processing by the photoacoustic apparatus according to the present exemplary embodiment will be described below. A case illustrated in FIG. 17 is considered here. More specifically, as illustrated in FIG. 17, the first light irradiation unit 310a emits intensity modulated light to irradiate the first irradiation region 381a with the light. As illustrated in FIG. 17, the second light irradiation unit 310b emits intensity modulated light to irradiate the second irradiation region 381b with the light. As illustrated in FIG. 17, the third light irradiation unit 310c emits intensity modulated light to irradiate the third irradiation region 381c with the light. A case is assumed where the subject 180 includes optical absorbers 390a, 390b, and 390c. The first optical absorber 390a exists near the surface of the subject 180 inside the first irradiation region 381a. The second optical absorber 390b exists near the surface of the subject 180 inside the second irradiation region 381b. The third optical absorber 390c exists near the surface of the subject 180 inside the third irradiation region 381c. The ratio of the absorption coefficients of the first optical absorber 390a, the second optical absorber 390b, and the third optical absorber 390c with respect to light with a wavelength of 808 nm is set to 1:0.5:0.75. The distance from the reception unit 120 to each optical absorber is assumed to be equal.

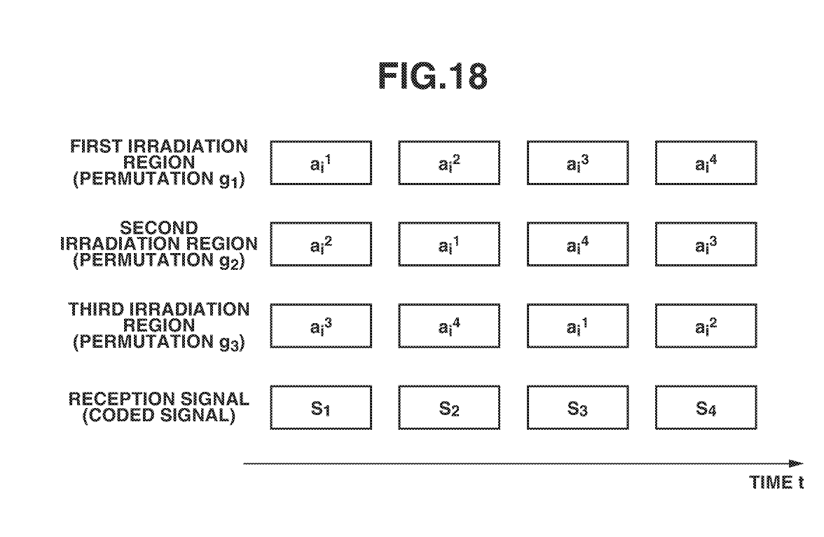

According to the present exemplary embodiment, in a sequence illustrated in FIG. 18, the computer 150 synchronously irradiates the subject 180 with intensity modulated light of the elements with the same number for respective permutations to perform coding processing.

For the first permutation elements, the light irradiation unit 110 synchronously radiates intensity modulated light corresponding to the first coding sequence {aᵢ¹}, intensity modulated light corresponding to the second coding sequence {aᵢ¹}, and intensity modulated light corresponding to the third coding sequence {aᵢ³}. In this case, the intensity modulated light corresponding to the first coding sequence {aᵢ¹} is radiated to the first irradiation region 381a, the intensity modulated light corresponding to the second coding sequence {aᵢ²} is radiated to the second irradiation region 381b, and the intensity modulated light corresponding to the third coding sequence {aᵢ³} is radiated to the third irradiation region 381c. Then, the reception unit 120 receives photoacoustic waves generated due to the light irradiations, and outputs a reception signal S₁.

For the second permutation elements, the light irradiation unit 110 synchronously radiates intensity modulated light corresponding to the second coding sequence {aᵢ²}, intensity modulated light corresponding to the first coding sequence {aᵢ¹}, and intensity modulated light corresponding to the fourth coding sequence {aᵢ⁴}. In this case, the intensity modulated light corresponding to the second coding sequence {aᵢ²} is radiated to the first irradiation region 381a, the intensity modulated light corresponding to the first coding sequence {aᵢ¹} is radiated to the second irradiation region 381b, and the intensity modulated light corresponding to the fourth coding sequence {aᵢ⁴} is radiated to the third irradiation region 381c. Then, the reception unit 120 receives photoacoustic waves generated due to the light irradiations, and outputs a reception signal S₂.

For the third permutation elements, the light irradiation unit 110 synchronously radiates intensity modulated light corresponding to the third coding sequence {aᵢ³}, intensity modulated light corresponding to the fourth coding sequence {aᵢ⁴}, and intensity modulated light corresponding to the first coding sequence {aᵢ¹}. In this case, the intensity modulated light corresponding to the third coding sequence {aᵢ³} is radiated to the first irradiation region 381a, the intensity modulated light corresponding to the fourth coding sequence {aᵢ⁴} is radiated to the second irradiation region 381b, and the intensity modulated light corresponding to the first coding sequence {aᵢ¹} is radiated to the third irradiation region 381c. Then, the reception unit 120 receives photoacoustic waves generated due to the light irradiations, and outputs a reception signal S₃.

For the fourth permutation elements, the light irradiation unit 110 synchronously radiates intensity modulated light corresponding to the fourth coding sequence {aᵢ⁴}, intensity modulated light corresponding to the third coding sequence {aᵢ³}, and intensity modulated light corresponding to the second coding sequence {aᵢ¹}. In this case, the intensity modulated light corresponding to the fourth coding sequence {aᵢ⁴} is radiated to the first irradiation region 381a, the intensity modulated light corresponding to the third coding sequence {aᵢ³} is radiated to the second irradiation region 381b, and the intensity modulated light corresponding to the second coding sequence {aᵢ²} is radiated to the third irradiation region 381c. Then, the reception unit 120 receives photoacoustic waves generated due to the light irradiations, and outputs a reception signal S₄.

The light irradiations to respective irradiation regions for respective permutation elements may be performed without complete synchronization. However, to improve the S/N ratio of signals acquired per unit time, it is desirable that the periods of intensity modulated light irradiations to a plurality of irradiation regions at least partially overlap.

### <Step S1. Intensity Modulated Light Irradiations Corresponding to First Permutation Elements>

The control unit 153 transmits the information about the first coding sequence {aᵢ¹} to the first drive unit 311a according to the assigned permutation. The control unit 153 also transmits the information about the second coding sequence {aᵢ¹} to the second drive unit 311b according to the assigned permutation. The control unit 153 also transmits the information about the third coding sequence {aᵢ³} to the third drive unit 311c according to the assigned permutation.

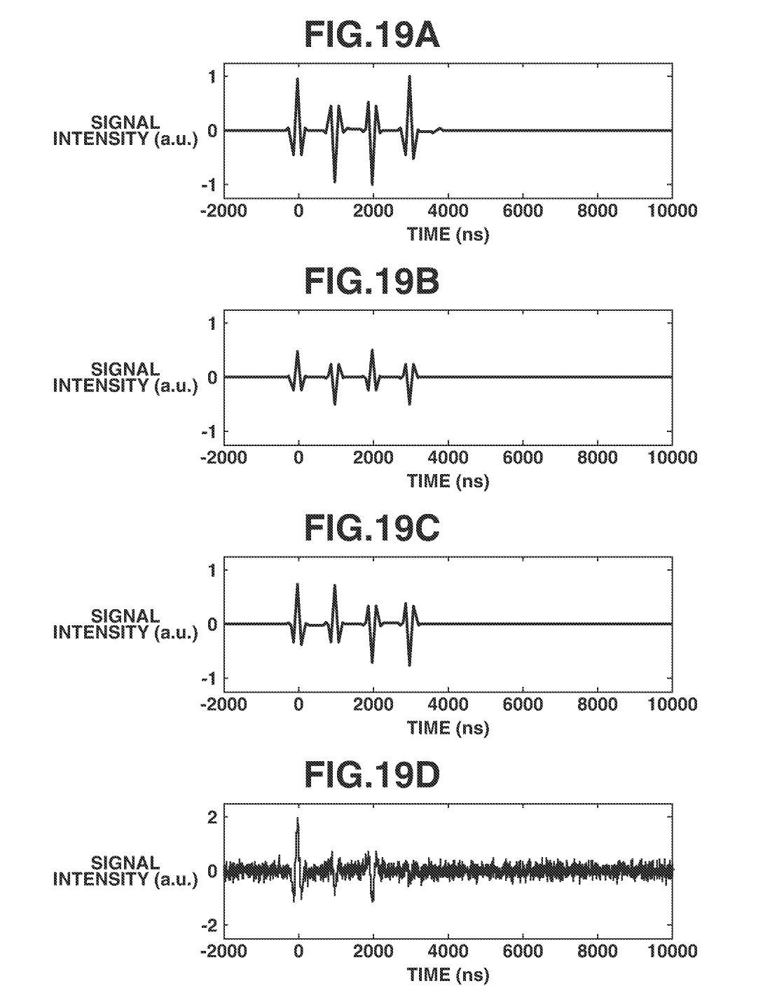

The first light source 312a is driven by a drive current generated by the first drive unit 311a based on the information about the first coding sequence {aᵢ¹}. The generated light is radiated to the point optical absorber (sound source) 390a via the first optical system 313a. Then, the reception unit 120 receives the generated photoacoustic wave. A reception signal acquired in this way has a waveform as illustrated in FIG. 19A.

The second light source 312b is driven by a drive current generated by the second drive unit 311b based on the information about the second coding sequence {aᵢ¹}. The generated light is radiated to the point optical absorber (sound source) 390b via the second optical system 313b. Then, the reception unit 120 receives the generated photoacoustic wave. A reception signal acquired in this way has a waveform as illustrated in FIG. 19B.

The third light source 312c is driven by a drive current generated by the third drive unit 311c based on the information about the third coding sequence {aᵢ³}. The generated light is radiated to the point optical absorber (sound source) 390c via the third optical system 313c. Then, the reception unit 120 receives the generated photoacoustic wave. A reception signal acquired in this way has a waveform as illustrated in FIG. 19C.

### <Step S2. Acquisition of Reception Signal Corresponding to First Permutation Elements>

When the optical outputs of the first light source 312a, the second light source 312b, and the third light source 312c are synchronized with each other (when these light sources emit light at approximately the same timing), the reception signal acquired when the reception unit 120 receives the generated photoacoustic waves is the sum of the signals illustrated in FIGS. 19A, 19B, and 19C. A reception signal S₁(t) acquired at this time is illustrated in FIG. 19D. More specifically, the reception signal S₁(t) is acquired by radiating the intensity modulated light corresponding to the first coding sequence {aᵢ¹}, the intensity modulated light corresponding to the second coding sequence {aᵢ¹}, and the intensity modulated light corresponding to the third coding sequence {aᵢ³}, at approximately the same timing. In this case, the intensity modulated light corresponding to the first coding sequence {aᵢ¹} is radiated to the first irradiation region 381a, the intensity modulated light corresponding to the second coding sequence {aᵢ¹} is radiated to the second irradiation region 381b, and the intensity modulated light corresponding to the third coding sequence {aᵢ³} is radiated to the third irradiation region 381c. To describe the effect of noise suppression, noise with an average value of 0 and a standard deviation of 0.2 is added.

### <Step S3. Intensity Modulated Light Irradiations Corresponding to Second Permutation Elements>

The control unit 153 transmits the information about the second coding sequence {aᵢ¹} to the first drive unit 311a according to the assigned permutation. The control unit 153 also transmits the information about the first coding sequence {aᵢ¹} to the second drive unit 311b according to the assigned permutation. The control unit 153 also transmits the information about the fourth coding sequence {aᵢ⁴} to the third drive unit 311c according to the assigned permutation.

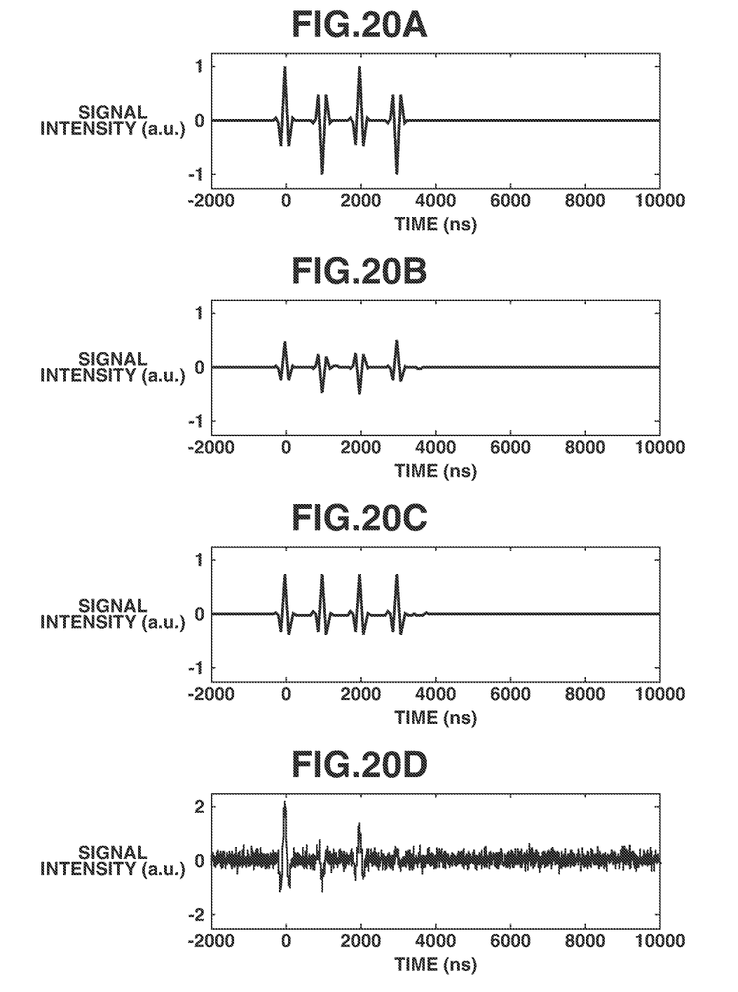

The first light source 312a is driven by a drive current generated by the first drive unit 311a based on the information about the second coding sequence {aᵢ¹}. The generated light is radiated to the point optical absorber (sound source) 390a via the first optical system 313a. Then, the reception unit 120 receives the generated photoacoustic wave. A reception signal acquired in this way has a waveform as illustrated in FIG. 20A.

The second light source 312b is driven by a drive current generated by the second drive unit 311b based on the information about the first coding sequence {aᵢ¹}. The generated light is radiated to the point optical absorber (sound source) 390b via the second optical system 313b. Then, the reception unit 120 receives the generated photoacoustic wave. A reception signal acquired in this way has a waveform as illustrated in FIG. 20B.

The third light source 312c is driven by a drive current generated by the third drive unit 311c based on the information about the fourth coding sequence {aᵢ⁴}. The generated light is radiated to the point optical absorber (sound source) 390c via the third optical system 313c. Then, the reception unit 120 receives the generated photoacoustic wave. A reception signal acquired in this way has a waveform as illustrated in FIG. 20C.

### <Step S4. Acquisition of Reception Signal Corresponding to Second Permutation Elements>

When the optical outputs of the first light source 312a, the second light source 312b, and the third light source 312c are synchronized with each other (when these light sources emit light at approximately the same timing), the reception signal acquired when the reception unit 120 receives the generated photoacoustic waves is the sum of the signals illustrated in FIGS. 20A, 20B, and 20C. A reception signal S₂(t) acquired at this time is illustrated in FIG. 20D. More specifically, the reception signal S₂(t) is acquired by radiating the intensity modulated light corresponding to the second coding sequence {aᵢ¹}, the intensity modulated light corresponding to the first coding sequence {aᵢ¹}, and the intensity modulated light corresponding to the fourth coding sequence {aᵢ⁴}, at approximately the same timing. In this case, the intensity modulated light corresponding to the second coding sequence {aᵢl} is radiated to the first irradiation region 381a, the intensity modulated light corresponding to the first coding sequence {aᵢ¹} is radiated to the second irradiation region 381b, and the intensity modulated light corresponding to the fourth coding sequence {aᵢ⁴} is radiated to the third irradiation region 381c. To describe the effect of noise suppression, noise with an average value of 0 and a standard deviation of 0.2 is added.

### <Step S5. Intensity Modulated Light Irradiations Corresponding to Third Permutation Elements>

The control unit 153 transmits the information about the third coding sequence {aᵢ³} to the first drive unit 311a according to the assigned permutation. The control unit 153 also transmits the information about the fourth coding sequence {aᵢ⁴} to the second drive unit 311b according to the assigned permutation. The control unit 153 also transmits the information about the first coding sequence {aᵢ¹} to the third drive unit 311c according to the assigned permutation.

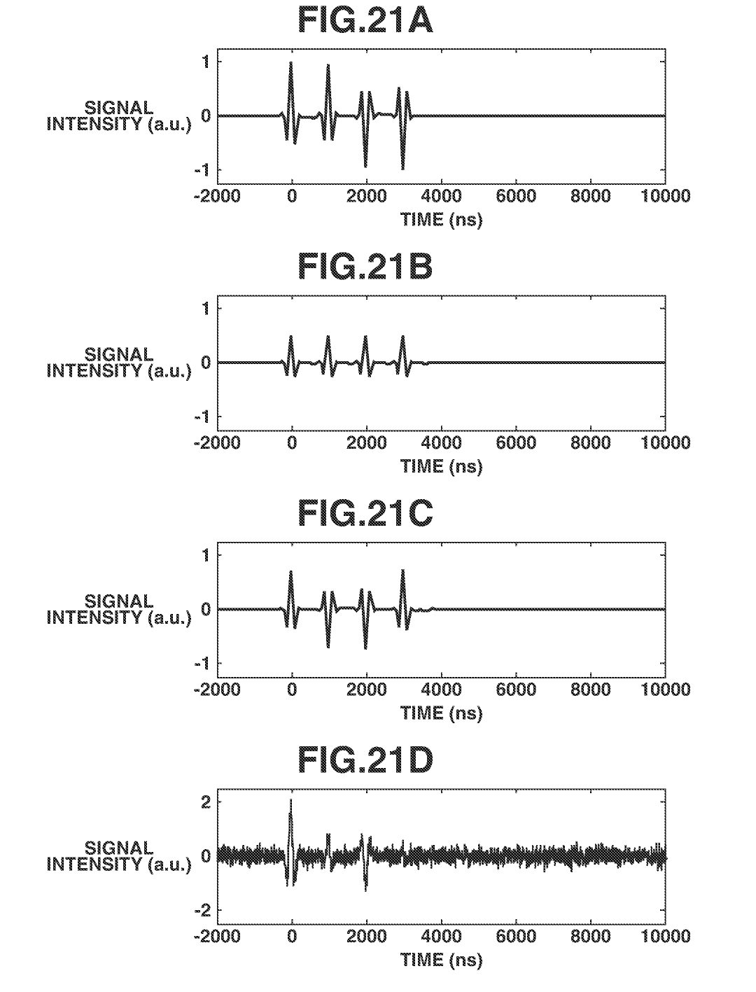

The first light source 312a is driven by a drive current generated by the first drive unit 311a based on the information about the third coding sequence {aᵢ³}. The generated light is radiated to the point optical absorber (sound source) 390a via the first optical system 313a. Then, the reception unit 120 receives the generated photoacoustic wave. A reception signal acquired in this way has a waveform as illustrated in FIG. 21A.

The second light source 312b is driven by a drive current generated by the second drive unit 311b based on the information about the fourth coding sequence {aᵢ⁴}. The generated light is radiated to the point optical absorber (sound source) 390b via the second optical system 313b. Then, the reception unit 120 receives the generated photoacoustic wave. A reception signal acquired in this way has a waveform as illustrated in FIG. 21B.

The third light source 312c is driven by a drive current generated by the third drive unit 311c based on the information about the first coding sequence {aᵢ¹}. The generated light is radiated to the point optical absorber (sound source) 390c via the third optical system 313c. Then, the reception unit 120 receives the generated photoacoustic wave. A reception signal acquired in this way has a waveform as illustrated in FIG. 21C.

### <Step S6. Acquisition of Reception Signal Corresponding to Third Permutation Elements>

When the optical outputs of the first light source 312a, the second light source 312b, and the third light source 312c are synchronized with each other (when these light sources emit light at approximately the same timing), the reception signal acquired when the reception unit 120 receives the generated photoacoustic waves is the sum of the signals illustrated in FIGS. 21A, 21B, and 21C. A reception signal S₃(t) acquired at this time is illustrated in FIG. 21D. More specifically, the reception signal S₃(t) is acquired by radiating the intensity modulated light corresponding to the third coding sequence {aᵢ³}, the intensity modulated light corresponding to the fourth coding sequence {aᵢ⁴}, and the intensity modulated light corresponding to the first coding sequence {aᵢ¹}, at approximately the same timing. In this case, the intensity modulated light corresponding to the third coding sequence {aᵢ³} is radiated to the first irradiation region 381a, the intensity modulated light corresponding to the fourth coding sequence {aᵢ⁴} is radiated to the second irradiation region 381b, and the intensity modulated light corresponding to the first coding sequence {aᵢ¹} is radiated to the third irradiation region 381c. To describe the effect of noise suppression, noise with an average value of 0 and a standard deviation of 0.2 is added.

### <Step S7. Intensity Modulated Light Irradiations Corresponding to Fourth Permutation Elements>

The control unit 153 transmits the information about the fourth coding sequence {aᵢ⁴} to the first drive unit 311a according to the assigned permutation. The control unit 153 also transmits the information about the third coding sequence {aᵢ³} to the second drive unit 311b according to the assigned permutation. The control unit 153 also transmits the information about the second coding sequence {aᵢ¹} to the third drive unit 311c according to the assigned permutation.

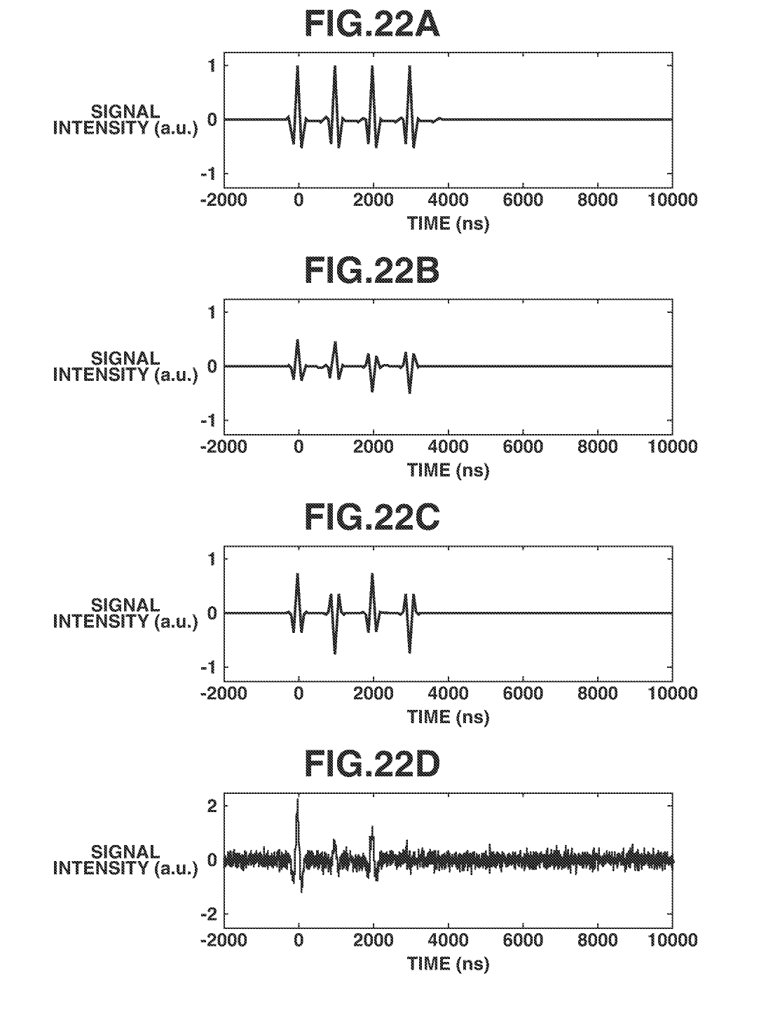

The first light source 312a is driven by a drive current generated by the first drive unit 311a based on the information about the fourth coding sequence {aᵢ⁴}. The generated light is radiated to the point optical absorber (sound source) 390a via the first optical system 313a. Then, the reception unit 120 receives the generated photoacoustic wave. A reception signal acquired in this way has a waveform as illustrated in FIG. 22A.

The second light source 312b is driven by a drive current generated by the second drive unit 311b based on the information about the third coding sequence {aᵢ³}. The generated light is radiated to the point optical absorber (sound source) 390b via the second optical system 313b. Then, the reception unit 120 receives the generated photoacoustic wave. A reception signal acquired in this way has a waveform as illustrated in FIG. 22B.

The third light source 312c is driven by a drive current generated by the third drive unit 311c based on the information about the second coding sequence {aᵢ¹}. The generated light is radiated to the point optical absorber (sound source) 390c via the third optical system 313c. Then, the reception unit 120 receives the generated photoacoustic wave. A reception signal acquired in this way has a waveform as illustrated in FIG. 22C.

### <Step S8. Acquisition of Reception Signal Corresponding to Fourth Permutation Elements>

When the optical outputs of the first light source 312a, the second light source 312b, and the third light source 312c are synchronized with each other (when these light sources emit light at approximately the same timing), the reception signal acquired when the reception unit 120 receives the generated photoacoustic waves is the sum of the signals illustrated in FIGS. 22A, 22B, and 22C. More specifically, the reception signal S₄(t) is acquired by radiating the intensity modulated light corresponding to the fourth coding sequence {aᵢ⁴}, the intensity modulated light corresponding to the third coding sequence {aᵢ³}, and the intensity modulated light corresponding to the second coding sequence {aᵢ¹}, at approximately the same timing. In this case, the intensity modulated light corresponding to the fourth coding sequence {aᵢ⁴} is radiated to the first irradiation region 381a, the intensity modulated light corresponding to the third coding sequence {aᵢ³} is radiated to the second irradiation region 381b, and the intensity modulated light corresponding to the second coding sequence {aᵢ²} is radiated to the third irradiation region 381c. A reception signal S₄(t) acquired at this time is illustrated in FIG. 22D. To describe the effect of noise suppression, noise with an average value of 0 and a standard deviation of 0.2 is added.

Referring to FIGS. 19 to 22, although the time actually shifts by the time period of photoacoustic wave propagation from each point optical absorber to the reception unit 120, this time shift is ignored.

### <Step S9. Decoding>

A method for decoding a coded reception signal performed by the calculation unit 151 in the computer 150 will be described below.

When Δt denotes the time interval between reference timings, the calculation unit 151 performs decoding processing according to the Formulas 9 to 11 to acquire the decoded signals DS₁(t), DS₂(t), and DS₃(t) for the intensity modulated light radiated to respective irradiation regions. The calculation unit 151 uses four different coding sequences for the decoding processing in the order determined by the same permutations as the ones assigned to light of the first, the second, and the third irradiation regions.

$$\begin{matrix}{DS}_{1} ( t ) = {\sum }_{i = 1}^{N} {S}_{1} ( t + ( i - 1 ) Δ t ) \cdot {a}_{i}^{1} + {\sum }_{i = 1}^{N} {S}_{2} ( t + ( i - 1 ) Δ t ) \cdot {a}_{i}^{2} + {\sum }_{i = 1}^{N} {S}_{3} ( t + ( i - 1 ) Δ t ) \cdot {a}_{i}^{3} + {\sum }_{i = 1}^{N} {S}_{4} ( t + ( i - 1 ) Δ t ) \cdot {a}_{i}^{4} & ( Formula 9 ) \\ {DS}_{2} ( t ) = {\sum }_{i = 1}^{N} {S}_{1} ( t + ( i - 1 ) Δ t ) \cdot {a}_{i}^{2} + {\sum }_{i = 1}^{N} {S}_{2} ( t + ( i - 1 ) Δ t ) \cdot {a}_{i}^{1} + {\sum }_{i = 1}^{N} {S}_{3} ( t + ( i - 1 ) Δ t ) \cdot {a}_{i}^{4} + {\sum }_{i = 1}^{N} {S}_{4} ( t + ( i - 1 ) Δ t ) \cdot {a}_{i}^{3} & ( Formula 10 ) \\ {DS}_{3} ( t ) = {\sum }_{i = 1}^{N} {S}_{1} ( t + ( i - 1 ) Δ t ) \cdot {a}_{i}^{3} + {\sum }_{i = 1}^{N} {S}_{2} ( t + ( i - 1 ) Δ t ) \cdot {a}_{i}^{4} + {\sum }_{i = 1}^{N} {S}_{3} ( t + ( i - 1 ) Δ t ) \cdot {a}_{i}^{1} + {\sum }_{i = 1}^{N} {S}_{4} ( t + ( i - 1 ) Δ t ) \cdot {a}_{i}^{2} & ( Formula 11 )\end{matrix}$$

When N denotes the code length, and K denotes the number of coding sequences which are orthogonal to each other (i.e., the number of permutation elements), generalizing the Formulas 9 to 11 gives Formula 12.

$${DS}_{m} ( t ) = {\sum }_{j = 1}^{K} {\sum }_{i = 1}^{N} {S}_{j} ( t + ( i - 1 ) Δ t ) \cdot {a}_{i}^{{g}_{m} ( j )} \qquad ( Formula 12 )$$

where, DSₘ denotes a decoded signal, i denotes a natural number of 1 or more, {gₘ(j)} denotes a permutation assigned to light to each of a plurality of irradiation regions, Sⱼ denotes a reception signal corresponding to a permutation element, and j denotes a natural number of 1 or more, and K denotes the power of 2 satisfying K≥M. In addition, m denotes a natural number of 1 or more and M or less, M denotes the number of irradiation regions, t denotes time, and Δt denotes the time interval between reference timings of coding elements in a coding sequence.

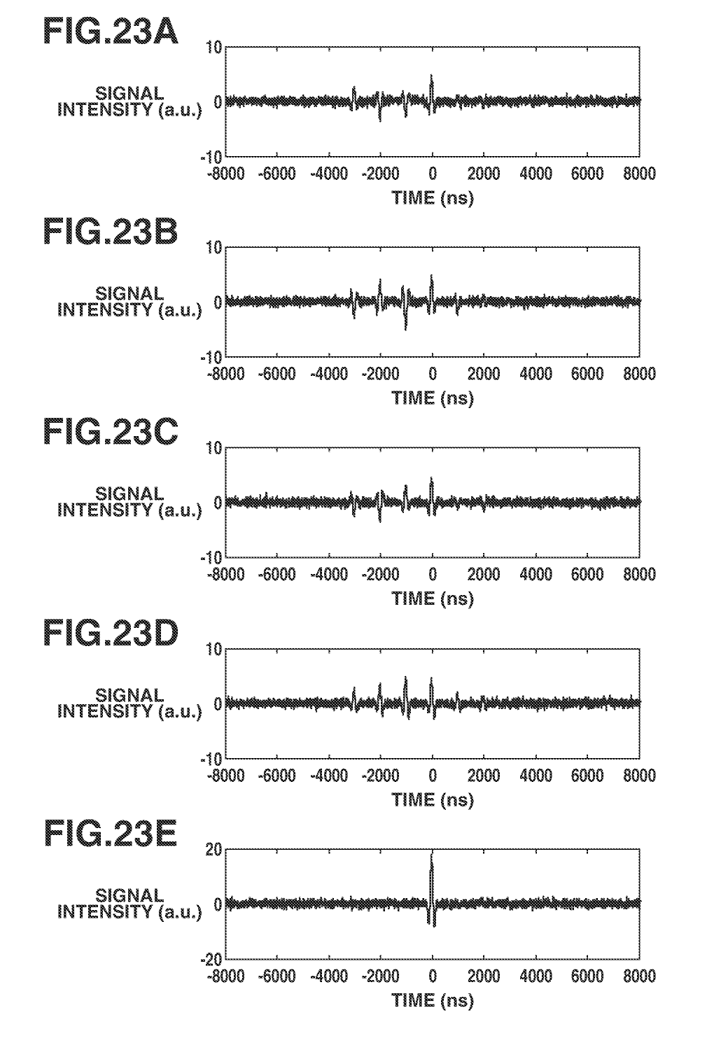

Decoding states corresponding to the first irradiation region 381a will be described below with reference to FIGS. 23A, 23B, 23C, 23D, and 23E.

As a result of performing decoding processing of the first term of the right-hand side of the Formula 9 on the reception waveform (S₁) illustrated in FIG. 19D, a decoded reception signal as illustrated in FIG. 23A can be acquired. As a result of performing decoding processing of the second term of the right-hand side of the Formula 9 on the reception waveform (S₂) illustrated in FIG. 20D, a decoded reception signal as illustrated in FIG. 23B can be acquired. As a result of performing decoding processing of the third term of the right-hand side of the Formula 9 on the reception waveform (S₄) illustrated in FIG. 21D, a decoded reception signal as illustrated in FIG. 23C can be acquired. As a result of performing decoding processing of the fourth term of the right-hand side of the Formula 9 on the reception waveform (S₄) illustrated in FIG. 22D, a decoded reception signal as illustrated in FIG. 23D can be acquired. The sum of the signals illustrated in FIGS. 23A, 23B, 23C, and 23D is the signal illustrated in FIG. 23E which indicates the decoded signal DS₁ corresponding to the intensity modulated light radiated to the first irradiation region 381a. More specifically, the peak value of the signal DS₁ decoded by the Formula 9 is increased 16 times the peak value of a reception signal acquired in one light irradiation, and side lobes are restrained to noise level or below.

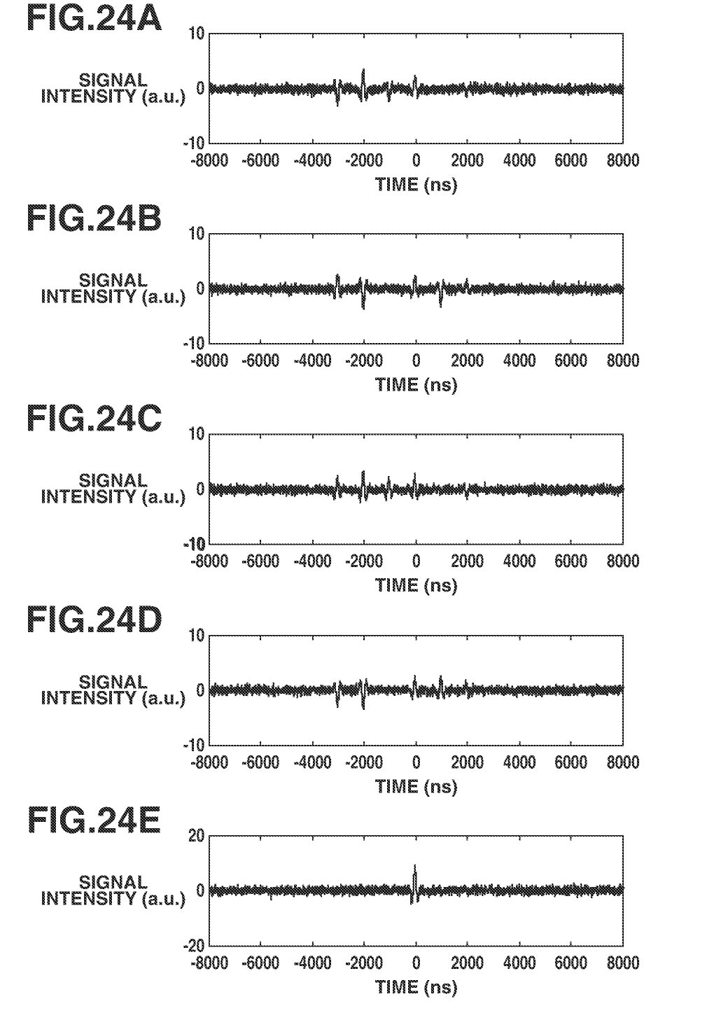

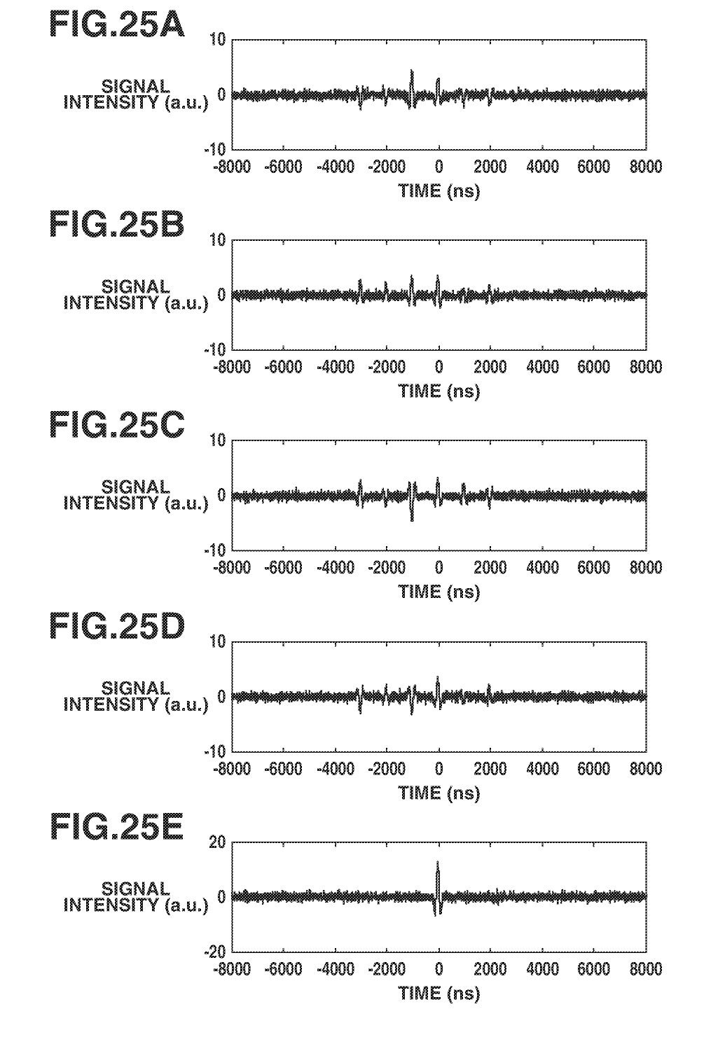

Likewise, decoding states corresponding to light irradiation on the second irradiation region 381b are illustrated in FIGS. 24A, 24B, 24C, 24D, and 24E, and decoding states corresponding to light irradiation on the third irradiation region 381c are illustrated in FIGS. 25A, 25B, 25C, 25D, and 25E. More specifically, the peak value of the signals DS₂ or DS₃ decoded by the Formulas 10 and 11, respectively, is increased 16 times the peak value of a reception signal acquired in one light irradiation, and side lobes are restrained to noise level or below.

When the signals illustrated in FIGS. 23E, 24E, and 25E are compared, the ratio of the absorption coefficients of absorbers is preserved. This means that information about the optical absorption of the subject 180 included in a reception signal is preserved even after coding and decoding processing is performed. Therefore, analyzing the decoded signals DS₁, DS₂, and DS₃ corresponding to intensity modulated light irradiated to a plurality of irradiation regions enables obtaining an optical absorption coefficient distribution in the subject 180.

Similar to the first exemplary embodiment, analyzing the decoded signals DS₁, DS₂, and DS₃ enables generating a plurality of photoacoustic images for respective irradiation regions. The computer 150 can display images of respective irradiation regions in a superimposed manner, in a parallelly arranged manner, or in a switched manner. In this way, the present exemplary embodiment makes it possible to independently perform display control on images respectively corresponding to a plurality of irradiation regions.

Although, in the present exemplary embodiment, decoded signals corresponding to respective three irradiation regions are acquired, a decoded signal corresponding to at least one of the three irradiation regions only needs to be acquired. More specifically, according to the present exemplary embodiment, a decoded signal corresponding to at least one of a plurality of irradiation regions only needs to be acquired. Also in this case, decoded signals corresponding to desired irradiation regions can be acquired.

Similar to the first exemplary embodiment, the control unit 153 may set the time interval between reference timings so as to acquire a signal with a high S/N ratio in a short time according to the observation region (target region) and the sound speed in the subject 180. A reconstruction method similar to the one according to the first exemplary embodiment is also applicable. The configuration of the drive units according to the first exemplary embodiment may also be applied to the present exemplary embodiment.

Although, in the present exemplary embodiment, three irradiation regions horizontally arranged are used, the arrangement of a plurality of irradiation regions may be in any form as long as mutually different regions are irradiated with light. For example, a plurality of irradiation regions may be formed in concentric ring shapes with different radii.

<Coding and Decoding Processing with Small Number of Coding Sequences>

In the method according to the second exemplary embodiment, for example, setting the code length to 8 instead of 4 enables improving the S/N ratio. However, in this case, the number of coding sequences orthogonal to each other is 8, and light irradiation and photoacoustic wave reception for coding need to be repeated 8 times, resulting in an increased measurement time.

The third exemplary embodiment will be described below centering on a case where decreasing the number of coding sequences improves the S/N ratio while restraining the increase in measurement time.

Four different coding sequences {aᵢ^{k}} (k=1 to 4, i=1 to 8) with a code length of 8, which are orthogonal to each other, are considered here.

First coding sequence {aᵢ¹}={1,1,−1,−1,−1,−1,1,1}
Second coding sequence {aᵢ²}={1,1,−1−1,1,1,−1,−1}
Third coding sequence {aᵢ³}={1,1,1,1, −1,−1,−1,−1}
Four coding sequence {aᵢ⁴}={1,1,1,1,1,1,1,1}
{1,1} and {−1,−1} are respectively assigned to {1} and {−1} of each coding sequence according to the second exemplary embodiment. More specifically, the number of coding elements is doubled by repeating twice each coding element of four coding sequences which are orthogonal to each other. The number of repetitions of each coding element is an arbitrary value as long as the number is a natural number of 2 or more.

In the following description, four mutually different permutations representing the order of sequentially arranging the four coding sequences without duplication, are considered.

First permutation {gᵢ}={{aᵢ¹},{aᵢ²}, {aᵢ³}, {aᵢ⁴}}
Second permutation {g₂}={{aᵢ²}, {aᵢ¹}, {aᵢ⁴}, {aᵢ³}}
Third permutation {g₃}={{aᵢ³}, {aᵢ⁴}, {aᵢ¹},{aᵢ²}}
Fourth permutation {g₄}={{aᵢ⁴}, {aᵢ³}, {aᵢ²}, {aᵢ¹}}
As represented by the Formula 6, the sum total of cross-correlation functions when the four coding sequences are arranged according to the permutations gₚ and g_{q}, is considered.

When p=q, the Formula 6 represents the sum total of auto-correlation functions, i.e., 16, 32, and 16 at the peak and 0 at all non-peak points. For example, assume the following case:

(a¹*a¹)={1,−2,−1,−4,−3,−2,3,8,3,−2,−3,−4,−1,2,1}
(a²*a²)={−1,−2,1,4,−1,−6,1,8,1,−6,−1,4,1,−2,−1}
(a³*a³)={−1,−2,−3,−4,−1,2,5,8,5,2,−1,−4,−3,−2,−1}
(a⁴*a⁴)={1,2,3,4,5,6,7,8,7,6,5,4,3,2,1}
In this case, the following formula is given at an arbitrary point p:

$$\begin{matrix}\begin{matrix}{\sum }_{j = 1}^{4} ( {a}^{{g}_{p} ( j )} * {a}^{{g}_{p} ( j )} ) = ( {a}^{1} * {a}^{1} ) + ( {a}^{2} * {a}^{2} ) + ( {a}^{3} * {a}^{3} ) + \\ ( {a}^{4} * {a}^{4} ) \\ = \{ 0 , 0 , 0 , 0 , 0 , 0 , 16 , 32 , 16 , \\ 0 , 0 , 0 , 0 , 0 , 0 \}\end{matrix} & ( Formula 13 )\end{matrix}$$

When p≠q, the Formula 6 represents the sum total of cross-correlation functions, i.e., 0 at all points in all combinations with p≠q. For example, assume the following case where p=1 and q=2:

(a¹*a²)={−1,−2,1,4,1,−2,−1,0,1,2,−1,−4,−1,2,1}
(a²*a¹)={1,2,−1,−4,−1,2,1,0,−1,−2,1,4,1,−2,−1}
(a³*a⁴)={1,2,3,4,3,2,1,0,−1,−2,−3,−4,−3,−2,−1}
(a⁴*a³)={−1,−2,−3,−4,−3,−2,−1,0,1,2,3,4,3,2,1}
In this case, the following formula is given.

$$\begin{matrix}\begin{matrix}{\sum }_{j = 1}^{4} ( {a}^{{g}_{1} ( j )} * {a}^{{g}_{2} ( j )} ) = ( {a}^{1} * {a}^{2} ) + ( {a}^{2} * {a}^{1} ) + ( {a}^{3} * {a}^{4} ) + \\ ( {a}^{4} * {a}^{3} ) \\ = \{ 0 , 0 , 0 , 0 , 0 , 0 , 0 , 0 , 0 , 0 , 0 , 0 , \\ 0 , 0 , 0 \}\end{matrix} & ( Formula 14 )\end{matrix}$$

A case is considered where the above-described coding sequences and a permutation indicating the order are applied to a photoacoustic apparatus using light to a plurality of mutually different irradiation regions. In this case, even if light irradiation time periods overlap, it is possible to separately acquire a signal resulting from the intensity modulated light to a certain irradiation region and a signal resulting from the intensity modulated light to another irradiation region. Analyzing decoded signals corresponding to the intensity modulated light to a plurality of irradiation regions separated in this way enables generating images respectively corresponding to a plurality of irradiation regions. The computer 150 can display images of respective irradiation regions in a superimposed manner, in a parallelly arranged manner, or in a switched manner. In this way, the present exemplary embodiment enables independently performing display control of images respectively corresponding to a plurality of irradiation regions.

When Δt<<T_{tof}, the peak intensity of a decoded signal corresponding to each irradiation region can be increased without increasing the measurement time in comparison with the second exemplary embodiment. Although, in this case, side lobes also increase, a desired correction only needs to be applied to the decoded reception signal as required since side lobe patterns are known. For example, it is also possible to validate only signals equal to or larger than a certain threshold value out of decoded reception signals. Since decoded reception signals are known to have a signal ratio of 1:2:1 at intervals of Δt, it is also possible to superimpose a deconvolution filter for correcting the decoded reception signals to signals with a signal ratio of 0:1:0.

Although, in the present exemplary embodiment, decoded signals respectively corresponding to a plurality of irradiation regions are acquired, a decoded signal corresponding to at least one of the plurality of irradiation regions only needs to be acquired. Also in this case, decoded signals corresponding to desired irradiation regions can be acquired.

A fourth exemplary embodiment will be described below centering on a display control method for photoacoustic images in a case where decoded signals respectively corresponding to a plurality of irradiation regions are acquired, as described in the first to the third exemplary embodiments.

As described above, according to the present exemplary embodiment, the computer 150 can generate photoacoustic images respectively corresponding to a plurality of irradiation regions based on decoded signals respectively corresponding to a plurality of irradiation regions. Then, the computer 150 can display a plurality of photoacoustic images corresponding to the plurality of irradiation regions in a superimposed manner, in a parallelly arranged manner, or in a switched manner.

In this case, the computer 150 may also weight images respectively corresponding to a plurality of irradiation regions before displaying the images. The computer 150 may also change the weight for the image corresponding to a certain irradiation region and the weight for the image corresponding to another irradiation region before displaying the images in a superimposed manner. The computer 150 may also weight each position of each image so as to selectively superimpose regions having an image value higher than a threshold value out of images respectively corresponding to irradiation regions before displaying the images in a superimposed manner. The computer 150 may also selectively superimpose predetermined portions in images corresponding to respective irradiation regions before displaying the images in a superimposed manner. Not only changing the weight between images but also changing the weight in each image enables selectively superimposing portions with high image quality out of images respectively corresponding to irradiation regions. According to the present exemplary embodiment, since an image with a high S/N ratio is generated for each irradiation region through coding and decoding processing, the image for each irradiation region can be weighted.

In addition to displaying weighted images in a superimposed manner, weighted images may be displayed in a parallelly arranged manner or in a switched manner.

In addition, the user may specify the weight to be given to each image or each position in the image by using the input unit 170. The computer 150 can determine the weight of photoacoustic images respectively corresponding to a plurality of irradiation regions by using information indicating the weight determined according to a user instruction.

The computer 150 may also display a combined image (e.g., an image having undergone average processing or addition average processing) generated by combining a plurality of photoacoustic images respectively corresponding to a plurality of irradiation regions. Then, the computer 150 may determine, through image processing, a region with a high image value out of combined images and generate a combined image with a decreased weight of the photoacoustic image corresponding to an irradiation region irradiated with light. Typically, there is a tendency that a photoacoustic image provides high image values at optical absorbers (such as a body hair and a mole) existing on the surface of the subject 180. There is also a tendency that a photoacoustic image including such images includes noise resulting from photoacoustic waves generated from optical absorbers. Therefore, noise components included in a combined image can be reduced by decreasing the weight of the photoacoustic image corresponding to an irradiation region where these optical absorbers are irradiated with light. By using the input unit 170, the user may specify unnecessary images such as a mole and a body hair for the combined image displayed on the display unit 160. By using information indicating unnecessary images determined according to a user instruction, the computer 150 may determine the weight for the photoacoustic image corresponding to an irradiation region where the unnecessary images are irradiated with light. More specifically, the computer 150 may make the weight for the photoacoustic image corresponding to an irradiation region where unnecessary images are irradiated with light smaller than the weight for photoacoustic images other than the photoacoustic image before regenerating a combined image.

Although, in the first to the fourth exemplary embodiments, decoded signals respectively corresponding to a plurality of irradiation regions are acquired, a decoded signal corresponding to at least one of a plurality of irradiation regions may be acquired. Also in this case, when a plurality of irradiation regions is irradiated with light, decoded signals corresponding to desired irradiation regions can be acquired, making it possible to generate photoacoustic images corresponding to the desired irradiation regions.

### Other Exemplary Embodiments

The present invention is implemented also by performing the following processing. More specifically, software (a program) for implementing the functions of the above-described exemplary embodiments is supplied to a system or apparatus via a network or various types of storage media, and a computer (or CPU or micro processing unit (MPU)) of the system or apparatus reads and executes the program.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-116919, filed Jun. 14, 2017, which is hereby incorporated by reference herein in its entirety.

What is claimed is:

1. A photoacoustic apparatus comprising:
a light irradiation unit configured to irradiate a first irradiation region of a subject with first intensity modulated light corresponding to a first coding sequence, and to irradiate a second irradiation region of the subject with second intensity modulated light corresponding to a second coding sequence;
a reception unit configured to receive photoacoustic waves generated when the subject is irradiated with the first intensity modulated light and the second intensity modulated light, and to output a first signal; and
a processing unit configured to perform decoding processing on the first signal based on information on the first and the second coding sequences to acquire at least one of a first decoded signal corresponding to the first irradiation region and a second decoded signal corresponding to the second irradiation region.

2. The photoacoustic apparatus according to claim 1, wherein an irradiation period of the first intensity modulated light at least partially overlaps with an irradiation period of the second intensity modulated light.

3. The photoacoustic apparatus according to claim 1, wherein each of the first and the second coding sequences includes a positive coding element and a negative coding element.

4. The photoacoustic apparatus according to claim 3,
wherein the light irradiation unit includes a light source including a semiconductor laser or a light emitting diode and a drive unit configured to supply a drive current to the light source,
wherein the drive unit supplies a first drive current for generating positive intensity modulated light corresponding to the positive coding element to the light source in accordance with a reference timing corresponding to the positive coding element, and
wherein the drive unit supplies a second drive current for generating negative intensity modulated light corresponding to the negative coding element to the light source in accordance with a reference timing corresponding to the positive coding element.

5. The photoacoustic apparatus according to claim 4, wherein the drive unit includes a first drive unit configured to supply the first drive current to the light source, and a second drive unit configured to supply the second drive current to the light source.

6. The photoacoustic apparatus according to claim 4, wherein the light source includes a first light source composed of a semiconductor laser or a light emitting diode for emitting light to be radiated to the first irradiation region, and a second light source composed of a semiconductor laser or a light emitting diode for emitting light to be radiated to the second irradiation region.

7. The photoacoustic apparatus according to claim 1,
wherein the light irradiation unit radiates third intensity modulated light corresponding to a third coding sequence to the first irradiation region, and fourth intensity modulated light corresponding to a fourth coding sequence to the second irradiation region,
wherein the reception unit receives photoacoustic waves generated when the subject is irradiated with the third intensity modulated light and the fourth intensity modulated light, and outputs a second signal, and
wherein the processing unit performs decoding processing on the first and the second signals based on information on the first and the third coding sequences to acquire the first decoded signal corresponding to the first irradiation region; and
wherein the processing unit performs decoding processing on the first and the second signals based on information on the second and the fourth coding sequences to acquire the second decoded signal corresponding to the second irradiation region.

8. The photoacoustic apparatus according to claim 7,
wherein the first and the third coding sequences are a first complementary code having a complementary relation,
wherein the second and the fourth coding sequences are a second complementary code having a complementary relation, and
wherein a sum of a cross-correlation function of the first and the second coding sequences and a cross-correlation function of the third and the fourth coding sequences becomes 0.

9. The photoacoustic apparatus according to claim 7,
wherein an irradiation period of the first intensity modulated light at least partially overlaps with an irradiation period of the second intensity modulated light,
wherein an irradiation period of the third intensity modulated light at least partially overlaps with an irradiation period of the fourth intensity modulated light, and
wherein the irradiation periods of the first and the second intensity modulated light do not overlap with the irradiation periods of the third and the fourth intensity modulated light.

10. The photoacoustic apparatus according to claim 7, wherein the processing unit performs the decoding processing according to the following formulas to acquire the first decoded signal DS₁(t) and the second decoded signal DS₂(t):
$$\begin{matrix}{DS}_{1} ( t ) = {\sum }_{i = 1}^{N} {S}_{1} ( t + ( i - 1 ) Δ t ) \cdot {a}_{i} + {\sum }_{i = 1}^{N} {S}_{2} ( t + ( i - 1 ) Δ t ) \cdot {c}_{i} & ( Formula 1 ) \\ {DS}_{2} ( t ) = {\sum }_{i = 1}^{N} {S}_{1} ( t + ( i - 1 ) Δ t ) \cdot {b}_{i} + {\sum }_{i = 1}^{N} {S}_{2} ( t + ( i - 1 ) Δ t ) \cdot {d}_{i} & ( Formula 2 )\end{matrix}$$
where Δt denotes a time interval between reference timings corresponding to coding elements, {aᵢ}, {bᵢ}, {cᵢ}, and {dᵢ} (i=1 to N (N is an integer), N denotes a code length and, coding element are 1 and −1) denote the first, the second, the third, and the fourth coding sequences, respectively, S₁(t) denotes the first signal, and S₂(t) denotes the second signal.

11. A photoacoustic apparatus comprising:
a light irradiation unit configured to irradiate a plurality of irradiation regions with a plurality of intensity modulated light beams corresponding to a plurality of coding sequences;
a reception unit configured to receive photoacoustic waves generated when the plurality of intensity modulated light beams is radiated, and to output a signal; and
a processing unit configured to perform decoding processing on the signal based on information on the plurality of coding sequences to acquire a decoded signal corresponding to at least one of the plurality of irradiation regions.

12. The photoacoustic apparatus according to claim 11, wherein the processing unit performs the decoding processing on the signal in accordance with the following formula to acquire the decoded signal (DSₘ(t) denotes a decoded signal, m denotes a natural number of 1 or more and M or less, M denotes the number of irradiation regions, and t denotes time)
$${DS}_{m} ( t ) = {\sum }_{j = 1}^{K} {\sum }_{i = 1}^{N} {S}_{j} ( t + ( i - 1 ) Δ t ) \cdot {a}_{i}^{{g}_{m} ( j )} \qquad ( Formula 3 )$$
where {aᵢ^{k}} denotes K coding sequences, i denotes a natural number of 1 or more, N denotes a code length, Δt denotes a time interval between reference timings of coding elements in a coding sequence, {gₘ(j)} denotes a permutation having K elements for determining an order of the K coding sequences assigned to the plurality of respective irradiation regions, j denotes a natural number of 1 or more, K denotes the number of permutation elements represented by the power of 2 satisfying K≥M, and Sⱼ(t) denotes the signal corresponding to each permutation element.

13. The photoacoustic apparatus according to claim 11, wherein the plurality of coding sequences is orthogonal to each other.

14. The photoacoustic apparatus according to claim 11, wherein the processing unit generates at least one photoacoustic image corresponding to at least one of the plurality of irradiation regions based on the decoded signal corresponding to at least one of the plurality of irradiation regions and displays at least one photoacoustic image on a display unit.

15. The photoacoustic apparatus according to claim 11,
wherein the processing unit, acquires a plurality of decoded signals corresponding to the plurality of irradiation regions by applying the decoding processing on the signal based on the information on the plurality of coding sequences, generates a plurality of photoacoustic images corresponding to the plurality of irradiation regions based on the plurality of decoded signals, and displays, on a display unit, a combined image generated by weighting and combining the plurality of photoacoustic images.

16. The photoacoustic apparatus according to claim 11, wherein the processing unit acquires a plurality of decoded signals corresponding to the plurality of irradiation regions by applying the decoding processing on the signal based on the information on the plurality of coding sequences, generates a plurality of photoacoustic images corresponding to the plurality of irradiation regions based on the plurality of decoded signals, displays a combined image of the plurality of photoacoustic images on a display unit, determines, based on information indicating a region determined according to a user instruction for the combined image displayed on the display unit, a photoacoustic image corresponding to an irradiation region irradiated with light, and combines the plurality of weighted photoacoustic images, a weight for the photoacoustic image being made smaller than a weight for other photoacoustic images.

17. A coding apparatus comprising:
a light irradiation unit configured to irradiate a first irradiation region of a subject with first intensity modulated light corresponding to a first coding sequence, and to irradiate a second irradiation region of the subject with second intensity modulated light corresponding to a second coding sequence; and
a reception unit configured to receive photoacoustic waves generated when the subject is irradiated with the first and the second intensity modulated light, and to output a coded signal.

18. The coding apparatus according to claim 17, wherein an irradiation period of the first intensity modulated light at least partially overlaps with an irradiation period of the second intensity modulated light.

19. The coding apparatus according to claim 17, wherein each of the first and the second coding sequences includes a positive coding element and a negative coding element.

20. An information processing method, comprising:
performing decoding processing on a reception signal of photoacoustic waves generated by irradiating a plurality of irradiation regions of a subject with a plurality of intensity modulated light beams corresponding to a plurality of coding sequences to acquire a decoded signal; and
performing decoding processing on the reception signal based on information on the plurality of coding sequences to acquire the decoded signal corresponding to at least one of the plurality of irradiation regions.
